SEQUENCE LISTING (32 sequences)

SEQ ID NO: 1 - protein, 97 aa, Homo sapiens
LQVDIVPSQGEISVGESKFFLCQVAGDAKDKDISWFSPNGEKLTPNQQRISVVWNDDSSSTLTIYNANIDDAGIYKCVVTGEDGSESEATVNVKIFQ

SEQ ID NO: 2 - protein, 90 aa, synthetic construct
  VARIANT 27..32: note = X can be any naturally occurring amino acid
  VARIANT 79: note = X can be any naturally occurring amino acid
LQVDIVPSQGEISVGESKFFLCQVAGXXXXXXISWFSPNGEKLTPNQQRISVVWNDDSSSTLTIYNANIDDAGIYKCVVXEATVNVKIFQ

SEQ ID NO: 3 - protein, 97 aa, Bos taurus
LQVDIVPSQGEISVGESKFFLCQVAGDAKDKDISWFSPNGEKLTPNQQRISVVWNDDSSSTLTIYNANIDDAGIYKCVVTAEDGTESEATVNVKIFQ

SEQ ID NO: 4 - protein, 97 aa, Mus musculus
LQVDIVPSQGEISVGESKFFLCQVAGDAKDKDISWFSPNGEKLSPNQQRISVVWNDDDSSTLTIYNANIDDAGIYKCVVTAEDGTQSEATVNVKIFQ

SEQ ID NO: 5 - protein, 97 aa, Rattus rattus; identical to SEQ ID NO: 4

SEQ ID NO: 6 - protein, 97 aa, Gallus gallus
LQVDIVPSQGEISVGESKFFLCQVAGEAKYKDISWFSPNGEKLTPNQQRISVVRNDDFSSTLTIYNANIDDAGIYKCVVSSVEEGDSEATVNVKIFQ

SEQ ID NO: 7 - protein, 94 aa, Xenopus laevis
LEVNIVPDQGEISLGESKFFLCQVSGEATDISWYSPTGEKLLNQQQISVVKNDEYTSTLTIYNVSSQDAGIYKCVASSETEGESEGTVNLKIYQ

SEQ ID NO: 8 - protein, 94 aa, Xenopus laevis
LEVNIVPDQGEISLGESKFFLCQVSGEATDISWYSPTGEKLVTQQQISVVRSDDYTSTLTIYNASSQDAGIYKCVASNEAEGESEGTVNLKIYQ

SEQ ID NO: 9 - protein, 93 aa, Homo sapiens
LQVTISLSKVELSVGESKFFTCTAIGEPESIDWYNPQGEKIISTQRVVVQKEGVRSRLTIYNANIEDAGIYRCQATDAKGQTQEATVVLEIYQ

SEQ ID NO: 10 - protein, 93 aa, Mus musculus
LQVTISLSKVELSVGESKFFTCTAIGEPESIDWYNPQGEKIISTQRVMLQKEGVRSRLTIYNANIEDAGIYRCQATDAKGQTQEATVVLEIYQ

SEQ ID NO: 11 - protein, 103 aa, synthetic construct
LQVDIVPSQGEISVGESKFFLCQVAGAHTVESISWFSPNGEKLTPNQQRISVVWNDDSSSTLTIYNANIDDAGIYKCVVVASARRGFGWVYPHEATVNVKIFQ

SEQ ID NO: 12 - protein, 6 aa, synthetic construct
AHTVES

SEQ ID NO: 13 - protein, 14 aa, synthetic construct
VASARRGFGWVYPH

SEQ ID NO: 14 - DNA, 309 bp, synthetic construct
  source 1..309: mol_type = other DNA
ctgcaagtagatattgtgccgagccagggcgaaattagcgtgggcgaaagcaaatttttcttatgccaggtggcgggcgcgcatactgtttagagtatcagctggtttagcccgaacggtgaaaaactgaccccgaatcagcaacgtatcggcgttgtgtggaacgatgatagctctagtaccctgacgatttacaatgcgaacattgatgacgcgggcatttataaatgcgtggtagtggcgtcggctcgtcgtggttttgggtgggtttatccgcatgaagccaccgttaatgtgaaaatctttcag

SEQ ID NO: 15 - protein, 6 aa, Homo sapiens
DAKDKD

SEQ ID NO: 16 - protein, 8 aa, Homo sapiens
TGEDGSES

SEQ ID NO: 17 - DNA, 18 bp, synthetic construct
  source 1..18: mol_type = other DNA
tggttgaggttgtgccca

SEQ ID NO: 18 - DNA, 19 bp, synthetic construct
  source 1..19: mol_type = other DNA
ctcgtgggtttgcctcatc

SEQ ID NO: 19 - DNA, 18 bp, synthetic construct
  source 1..18: mol_type = other DNA
gcgagcaactgagaagac

SEQ ID NO: 20 - DNA, 18 bp, synthetic construct
  source 1..18: mol_type = other DNA
ttgaaacccgagaacatc

SEQ ID NO: 21 - DNA, 21 bp, synthetic construct
  source 1..21: mol_type = other DNA
caacgccctgaccaccgatag

SEQ ID NO: 22 - DNA, 21 bp, synthetic construct
  source 1..21: mol_type = other DNA
ggctgccttccgtctcatagt

SEQ ID NO: 23 - DNA, 22 bp, synthetic construct
  source 1..22: mol_type = other DNA
gtgagaccttggaagacctgaa

SEQ ID NO: 24 - DNA, 20 bp, synthetic construct
  source 1..20: mol_type = other DNA
gagaaatgtgctcaggggct

SEQ ID NO: 25 - DNA, 18 bp, synthetic construct
  source 1..18: mol_type = other DNA
tgtggccatctttatgct

SEQ ID NO: 26 - DNA, 18 bp, synthetic construct
  source 1..18: mol_type = other DNA
gtcatttctttggggctt

SEQ ID NO: 27 - DNA, 18 bp, synthetic construct
  source 1..18: mol_type = other DNA
gggagaaaaacctgaagc

SEQ ID NO: 28 - DNA, 18 bp, synthetic construct
  source 1..18: mol_type = other DNA
attctggggactcagagc

SEQ ID NO: 29 - DNA, 22 bp, synthetic construct
  source 1..22: mol_type = other DNA
cgtgtctggagattcgacttga

SEQ ID NO: 30 - DNA, 20 bp, synthetic construct
  source 1..20: mol_type = other DNA
ttggaaactcacacgccaga

SEQ ID NO: 31 - DNA, 24 bp, synthetic construct
  source 1..24: mol_type = other DNA
aagatcaagatcattgctcctcct

SEQ ID NO: 32 - DNA, 20 bp, synthetic construct
  source 1..20: mol_type = other DNA
agctcagtaacagtccgcct

(19) United States
(12) Patent Application Publication
(10) Pub. No.: US 2025/0297020 A1
(43) Pub. Date: Sep. 25, 2025

(54) RANK-L BINDING MOLECULES

(71) Applicant: ADALTA LIMITED, Bundoora (AU)

(72) Inventors: Michael Foley, South Yarra (AU); Jiake Xu, City Beach (AU); Heng Qiu, Perth (AU); Chris Hosking, Preston (AU); Kevin Kimhour Lim, Brunswick East (AU)

(21) Appl. No.: 18/713,040

(22) PCT Filed: Nov. 23, 2022
(86) PCT No.: PCT/AU2022/051402

§ 371 Date: May 23, 2024

(30) Foreign Application Priority Data
Nov. 23, 2021 (AU) 2021903772

(51) Int. Cl. C07K 16/28; A61P 19/10

(52) U.S. Cl. CPC C07K 16/2875; A61P 19/10; C07K 2317/33; C07K 2317/569; C07K 2317/76; C07K 2317/92; C07K 2317/94

(57) ABSTRACT

The present invention relates to polypeptides that are directed against Receptor Activator of Nuclear Factor Kappa B Ligand (RANK-L) also known as tumor necrosis factor ligand superfamily member 11 (TNFSF11), TNF-related activation-induced cytokine (TRANCE), osteoprotegerin ligand (OPGL), and osteoclast differentiation factor (ODF). In a preferred embodiment, the polypeptide is an i-body comprising the modified domain 1 of NCAM forming the i-body scaffold, and an antigen-binding domain comprising CDR1 and CDR3 based on shark IgNAR antibody.

### CROSS-REFERENCE TO RELATED APPLICATIONS

This application is a U.S. National Phase Patent Application filed under 35 U.S.C. § 371 of International Application Number PCT/AU2022/051402, filed on Nov. 23, 2022, which claims priority to and the benefit of Australian Patent Application Number 2021903772, filed on Nov. 23, 2021, the entire contents of all of which are incorporated herein by reference.

All documents cited or referenced herein, and all documents cited or referenced in herein cited documents, together with any manufacturer's instructions, descriptions, product specifications, and product sheets for any products mentioned herein or in any document incorporated by reference herein, are hereby incorporated herein by reference in their entirety.

### SEQUENCE LISTING

The instant application includes a Sequence Listing submitted in XML format, which Sequence Listing is hereby incorporated by reference in its entirety. The XML file was last modified on Dec. 13, 2024, is named 535441PCT sequence listing updated 13420825_1.xml, and is 30 Kilobytes in size.

### FIELD OF THE INVENTION

The present disclosure relates to polypeptides that are directed against Receptor Activator of Nuclear factor Kappa B Ligand (RANK-L) also known as tumor necrosis factor ligand superfamily member 11 (TNFSF11), TNF-related activation-induced cytokine (TRANCE), osteoprotegerin ligand (OPGL), and osteoclast differentiation factor (ODF). The invention also relates to nucleic acids encoding such polypeptides; to methods for preparing such polypeptides; to compositions and in particular to pharmaceutical compositions, that compromise such polypeptides and to uses of such polypeptides for therapeutic or diagnostic purposes.

### BACKGROUND OF THE INVENTION

The human skeleton is the second largest component of the body, comprising about 14.84% of total weight. It allows for locomotion, protects vital organs, stores minerals and produces blood cells as well as endocrine factors. Bone is constantly undergoing life-time remodelling which is a metabolic process of bone breakdown and bone formation. It is a process of gradual removal and replacement of bone that is performed respectively by osteoclasts and osteoblasts whose coordinated activity serve to renew the bone structure and maintain bone mass and strength. Interrupting the balance of this dynamic interaction between bone-resorptive cells, osteoclasts, and bone-forming cells, osteoblasts, leads to skeletal disorders like osteoporosis, osteopetrosis, Paget's disease, etc. Osteoporosis is defined as a chronic skeletal condition characterized by low bone mass and deteriorated microarchitecture of bone tissue, resulting in increased bone fragility and susceptibility to fracture, especially of the hip, spine, and wrist (Compston, J. E., McClung, M. R., and Leslie, W. D. (2019) Osteoporosis. Lancet 393, 364-376).

In Australia, the total cost relating to osteoporosis was $7.4 billion per annum and, it is estimated that by 2022, 6.2 million Australians over 50 years old will suffer osteoporosis or osteopenia, aggravating the health and socioeconomic burden. The prevalence of osteoporosis in China shows a similar trend with more than 60 million people in total (6.46% men and 29.13% women aged over 50) estimated to suffer from osteoporosis.

Bone resorption by osteoclasts is critically dependent on and regulated by the TNF superfamily member receptor activator of RANK-L. RANK-L is expressed in membrane bound form on osteoblasts and stromal cells, although it can be produced in soluble form by activated T cells, the latter possibly contributing to inflammation related bone loss. The binding of RANK-L to its receptor, RANK, which is expressed on progenitors and precursors of osteoclasts is a critical point of control for osteoclastogenesis and bone resorption. RANK-L-dependent signals thus been shown to play a central role in osteoporosis and cancer-induced bone destruction, but also in other pathologies, most notably breast cancer metastases in soft tissues.

Common anti-resorptive agents against osteoporosis include bisphosphonates and Denosumab (Prolia). Denosumab is the first FDA-approved humanized monoclonal antibody (IgG2) that antagonizes the receptor activator of nuclear factor NF-KB ligand (RANKL) and inhibits osteoclast differentiation (Tu, K. N., Lie, J. D., Wan, C. K. V., Cameron, M., Austel, A. G., Nguyen, J. K., Van, K., and Hyun, D. (2018) Osteoporosis: A Review of Treatment Options.PT 43, 92-104).

RANKL is a type II transmembrane glycoprotein produced by mesenchymal lineage that binds to its receptor, RANK, and induces osteoclast differentiation and bone resorption (Ono, T., Hayashi, M., Sasaki, F., and Nakashima, T. (2020) RANKL biology: bone metabolism, the immune system, and beyond. Inflamm Regen 40, 2). Excessive RANKL causes hyperactive osteoclasts and enhanced osteolysis commonly observed in osteoporosis, rheumatoid arthritis (RA) and cancer treatment-induced bone loss, suggesting that it is a druggable target. Denosumab was proven to reduce fracture incidence, however, limitations such as its large molecular weight, difficulty and high cost of manufacture, accelerated bone loss and spontaneous fractures following treatment discontinuation were reported, providing a strong need for next-generation biological therapeutic goods (Bone, H. G., Wagman, R. B., Brandi, M. L., Brown, J. P., Chapurlat, R., Cummings, S. R., Czerwinski, E., Fahrleitner-Pammer, A., Kendler, D. L., Lippuner, K., Reginster, J. Y., Roux, C., Malouf, J., Bradley, M. N., Daizadeh, N. S., Wang, A., Dakin, P., Pannacciulli, N., Dempster, D. W., and Papapoulos, S. (2017) 10 years of denosumab treatment in postmenopausal women with osteoporosis: results from the phase 3 randomised FREEDOM trial and open-label extension. Lancet Diabetes Endocrinol 5, 513-523).

Such complete antibodies however face the drawback of full size antibodies such as high productions costs, low stability and their large size, which for example reduces the potential for tumor penetration.

### SUMMARY OF THE DISCLOSURE

Variable new antigen receptors (VNARs), single-domain antibody-like molecules, are the variable regions of shark antibodies which possess exquisite stability and high affinity against specific antigens. The structure of VNARs is similar to the i-set family of immunoglobulin domains (Igs), e.g. the immunoglobulin domains of human neural cell adhesion molecule 1 (NCAM), suggesting that the NCAM lg domain can be potentially refitted as a human-compatible scaffold.

The present disclosure relates to polypeptides referred to herein as “i-bodies”. These i-bodies bind to RANK-L and can be used to block the interaction of RANK-L to its receptor RANK and therefore modulate or inhibit or prevent downstream signalling. The disruption of the RANK-L, RANK signaling may as an example result in the inhibition of differentiation or proliferation of osteoclasts, the resorption of bone and in the chemotaxis of cancer cell lines. The i-bodies of the present disclosure are therefore useful therapeutic agents for the treatment or prevention of bone related disorders such as osteoporosis and bone metastases in various forms of cancer.

It will be appreciated that the i-bodies of the present disclosure provide advantages over other similar polypeptides and molecules such as traditional antibodies. Like traditional antibodies, the i-bodies of the present disclosure are able to bind to their target with high affinity and high specificity but their smaller size and stability are advantageous when compared to traditional therapeutic antibodies, polypeptides or peptides. I-bodies are also more stable molecules than conventional antibodies which leads to alternative routes of administration and to lower dose form, less frequent dosage to less side effect. I-bodies are also smaller in size and therefore can penetrate tissues, organs and areas such as the bone matrix that other large proteins may not be able to penetrate.

Due to its relatively small size, the i-body is ideally suited for tailoring half-life which will have advantages with use as an imaging agent or in the delivery of a required dose for a set period of time. Due to the small size the i-body is also ideally suited for the generation of multivalent or multi-specific polypeptides, and therefore will be able to bind on respectively 2 or 3 sub-units of the trimer RANK-L molecule and might be advantageous because of their higher potency. As a small polypeptide, the i-body also provides delivery of a pay-load to a target through conjugation to the polypeptide.

The present disclosure provides a polypeptide comprising a scaffold region comprising a sequence at least 80% identical, or at least 85% identical to SEQ ID NO:11.

The present disclosure also provides a polypeptide which comprises a sequence derived from Domain 1 of NCAM comprising a scaffold region and CDR1 and CDR3 regions, wherein the CDR1 region of the sequence derived from Domain 1 of NCAM is replaced with a CDR 1 region comprising a sequence having at least 90% identity to SEQ ID NO: 12; and wherein the CDR3 region derived from Domain 1 of NCAM is replaced with a CDR 3 region comprising a sequence having at least 90% identity to SEQ ID NO: 13; and wherein the polypeptide binds to human RANK-L.

In one embodiment, the CDR1 region derived from Domain 1 of NCAM is replaced with a CDR 1 region comprising or consisting of a sequence having at least 95% identity, or at least 97% identity, or at least 98% identity, or at least 99% identity, or 100% identity to SEQ ID NO: 12.

In one embodiment, the CDR3 region derived from Domain 1 of NCAM is replaced with a CDR 3 region comprising or consisting of a sequence having at least 95% identity, or at least 97% identity, or at least 98% identity, or at least 99% identity, or 100% identity to SEQ ID NO: 13. In one example, the CDR3 region is between 10 and 20 amino acids in length. In another example, the CDR3 region is between 11 and 16 amino acids in length.

In one embodiment the scaffold region comprises a sequence at least 90% identical to a scaffold region defined by amino acids 1 to 26, 33 to 79 and 88 to 97 respectively of SEQ ID NO:1.

In one embodiment, the positions of the CDR1 and CDR3 regions in the polypeptide respectively correspond to amino acids 27-32 and 80-87 of SEQ ID NO:1.

In one example, the scaffold region comprises a sequence which has at least 50%, or at least 60%, or at least 70%, or at least 80%, or at least 90%, or at least 95%, or at least 97%, or at least 98%, or at least 99% identity, or 100% identity with SEQ ID NO:2.

In one example the scaffold region comprises a sequence which has at least 45%, or at least 50%, or at least 60%, or at least 70%, or at least 80%, or at least 90%, or at least 95%, or at least 97%, or at least 98%, or at least 99% identity, or 100% identity with SEQ ID NO:1 excluding the CDR1 and CDR3 regions.

In one example, the scaffold region comprises a sequence which has at least 70%, or at least 80%, or at least 90%, or at least 95%, or at least 97%, or at least 98%, or at least 99% homology with SEQ ID NO: 2. In one example, the scaffold region comprises the sequence of SEQ ID NO:2.

In one example the scaffold region comprises a sequence which has at least 70%, or at least 80%, or at least 90%, or at least 95%, or at least 97%, or at least 98%, or at least 99% homology with SEQ ID NO:1 excluding the CDR1 and CDR3 regions. In one example, the scaffold region comprises the sequence of SEQ ID NO:1 excluding the CDR1 and CDR3 regions corresponding to DAKDKD (SEQ ID NO:15) and TGEDGSES (SEQ ID NO:16) respectively.

In one example, the amino acid sequence or polypeptide binds to human RANK-L with an affinity (Ko) of 150 nM or less, such as 100 nM or less, 50 nM or less, 25 nM or less, 15 nM or less, 10 nM or less or 5 nM or less. In one embodiment the polypeptide binds to human RANK-L with affinity or avidity of less than or about 15 nM.

In one example, the Ko is between about 0.01 nM to about 15 nM, such as between about 0.05 nM to about 5 nM, for example, between about 0.1 nM to about 1 nM, for example, between about 0.5 nM to about 1 nM.

In one example, the Ko is assessed by immobilizing the human RANK-L and assessing binding of the polypeptide to the immobilized human RANK-L using surface plasmon resonance.

An exemplary polypeptide of the disclosure has a Ko of about 10 nM (e.g., +/−5 nM) for human RANK-L. In a particular example, the polypeptide has a Ko of about 13 nM.

In another example, the association rate (Ka) or the dissociation rate (Kd) is between about 5×10³M·¹s·¹ to about 5×10⁵M·¹s·¹, for example, between about 1×10⁴M·¹s·¹ to about 4×10⁵M·¹s·¹, for example, between about 2×10⁴M·¹s·¹ to about 4×10⁵M·¹s·¹. In one example, the Ka is assessed by immobilizing the human RANK-Land assessing binding of the molecule to the immobilized human RANK-L using surface plasmon resonance.

An exemplary polypeptide of the disclosure has a Ka of about 2.3×10⁴M·¹s·¹. A further exemplary binding molecule of the disclosure has a Kd of about 2.9×10⁴M·¹s·¹. In one example, the Ka and Kd are assessed by immobilizing the human RANK-Land assessing binding of the binding molecule to the immobilized human RANK-L using surface plasmon resonance.

In one example the polypeptide can be used to modulate (inhibit, prevent or boost) the differentiation and/or proliferation of osteoclasts. The differentiation and or proliferation may be increased or decreased by at least 30% preferably at least 50% or at least 75%, or 80% or 90% or more, compared to the differentiation and or proliferation of osteoclasts under the same condition without the presence of the polypeptide.

In another example the polypeptide can be used to inhibit osteoclast differentiation in an osteoclastogenesis assay with an ICso of less than 5 nM. In one example the inhibition of RANK-L induced osteoclastogenesis is determined by way of a TRAP assay using murine RAW 264 cells as described herein. In another example, the polypeptide inhibits osteoclastogenesis of bone marrow macrophages (BBM).

In another example the polypeptide of the invention can be used to modulate the resorption of bone. The resorption may be increased or decreased by at least 30% preferably at least 50%, or at least 60%, or at least 65%, or at least 70%, or at least 75%, or 80% or 90% or more, compared to the resorption of bone under the same condition without the presence of the polypeptide.

In another example the polypeptide of the invention has an effect on osteoclast differentiation or bone resorpotion and can be used in the treatment of bone diseases.

In another example the polypeptide of the invention has a cytotoxic effect and can be used in the treatment of bone mestastasis or metastatic bone diseases.

In one example the polypeptide comprises a sequence that has at least 80% identity, at least 90% identity, or at least 95% identity, or at least 97% identity, or at least 98% identity, or at least 99% identity to identity to SEQ ID NO: 11.

In one example the polypeptide comprises or consists of the sequence SEQ ID NO:11. In one example, the polypeptide comprises a CDR1 having the sequence set forth in SEQ ID NO: 12 (AHTVES) and a CRD3 having the sequence set forth in SEQ ID NO:13 (VASARRGFGWVYPH).

In one example the polypeptide of the present disclosure binds specifically to RANK-L. A polypeptide which binds specifically to RANK-L does not have any significant binding or affinity to related molecules CD40L, TNF-a, TGF-B, TRAIL, OPG, or the binding to any one of the related molecules is 1000 times lower than the affinity the polypeptide has for RANK-L. In a further example the polypeptide of the disclosure binds to human RANK-Land mouse RANK-L.

The polypeptide of the invention will generally bind to a number of forms of RANK-L including soluble, membrane bound, synthetic, or any other variants including monomeric, multimeric or any other associated forms.

In another example the polypeptide of the disclosure is PEGylated.

The present disclosure also provides a nucleic acid molecule encoding a polypeptide described herein.

In one example the nucleic acid molecule comprises a sequence that has at least 80% identity, at least 90% identity, or at least 95% identity, or at least 97% identity, or at least 98% identity, or at least 99% identity to identity to SEQ ID NO:14. In one example, the nucleic acid molecule comprises the sequence set forth in SEQ ID NO:14.

The present disclosure also provides an expression construct comprising the nucleic acid molecule described herein.

The present disclosure also provides a host cell comprising the nucleic acid molecule or expression construct described herein.

In another aspect, the present invention provides a method of producing a polypeptide of the disclosure which comprises culturing a host cell under conditions enabling expression of the polypeptide and recovering the polypeptide.

The present disclosure also provides a conjugate comprising a polypeptide described herein and an agent.

The agent may be, for example, a therapeutic agent, a toxin, a detectable label or an agent which extends the half-life of the polypeptide.

In one example the agent which extends the half-life of the polypeptide is a serum protein or an Fe portion of an immunoglobulin.

In another example the polypeptide of the invention may be linked to a toxin or cytotoxic drug for delivery to cells such as tumour cells.

In another example the polypeptide of the invention may be linked to a label such as a radioisotope.

The present disclosure also provides a multimer comprising two or more polypeptides described herein. The polypeptides may comprise the same or different amino acid sequences. For example, in its simplest form, at least two polypeptides are directly linked via a suitable linker or sequence or spacer. For example the linker or spacer can be between 1 and 50 amino acids. For example a suitable linker is a GS9 linker or GS15 linker or a GS20 linker.

The present disclosure also provides for multivalent or multispecific polypeptides. In one example the disclosure provides a polypeptide of the present disclosure linked to a polypeptide directed to a target other than RANK-L, including by not limited to human serum albumin to increase half-life, CD3, CD64, CD16 or CD89 to redirect and activate any circulating T cells against tumors.

The present disclosure also provides a pharmaceutical composition comprising a polypeptide or a conjugate or a multimer as described herein and an acceptable carrier.

The present disclosure also provides a method of treating a pathological condition associated with an imbalance in RANK-L signaling or a pathway or mechanism in which RANK-L is involved, comprising administering to a subject in need thereof a polypeptide or a nucleic acid molecule or a conjugate or a multimer of the present disclosure.

Such diseases and disorders include but are not limited to bone disorders including osteoporosis, inflammatory conditions, autoimmune conditions, asthma, rheumatoid arthritis, multiple myeloma, multiple sclerosis and bone metastasis.

The present disclosure additionally provides the polypeptide or the nucleic acid or the expression construct or the cell or the composition of the present disclosure for use in the treatment or prophylaxis of a RANK-L-mediated condition.

The present disclosure additionally provides for use of the polypeptide or the nucleic acid or the expression construct or the cell or the composition of the present disclosure in medicine.

The present disclosure additionally provides for use of the polypeptide or the nucleic acid or the expression construct or the cell of the present disclosure in the manufacture of a medicament for the treatment or prophylaxis of a RANK-L-mediated condition.

The present disclosure also provides a method of treating angiogenesis in a subject, comprising administering to the subject a polypeptide or a nucleic acid molecule or a conjugate or a multimer of the present disclosure. In one example, the angiogenesis is associated with osteoporosis. In one example, the angiogenesis is related to endothelial cell migration.

The polypeptide of the present disclosure can also be used in a diagnostic format.

The present disclosure therefore additionally provides a method for detecting RANK-L in a sample, the method comprising contacting a sample with the polypeptide of the disclosure such that a RANK-L-polypeptide complex forms and detecting the complex, wherein detecting the complex is indicative of RANK-L in the sample. In one example, the sample is from a subject suffering from a RANK-L-mediated condition.

The present disclosure additionally provides a method for diagnosing a RANK-L-mediated condition in a subject, the method comprising performing the method described herein for detecting RANK-Lin a sample from the subject, wherein detection of RANK-Lin the sample is indicative of the condition.

In one example, the method comprises determining the level of RANK-L in the sample, wherein an increased or decreased level of RANK-L in the sample compared to a control sample is indicative of the condition.

The present disclosure additionally provides a method for localizing and/or detecting and/or diagnosing and/or prognosing a RANK-L-mediated condition, the method comprising detecting in vivo the polypeptide of the present disclosure bound to RANK-L, if present, wherein the polypeptide is conjugated to a detectable tag.

In one example, the method additionally comprises administering the polypeptide to the subject.

In one example of any method of treatment/prophylaxis/diagnosis/prognosis described herein the RANK-L-mediated condition is a bone disorder such as osteoporosis, inflammatory conditions, autoimmune condition, asthma, rheumatoid arthritis, multiple myeloma, multiple sclerosis or bone metastasis.

### BRIEF DESCRIPTION OF THE FIGURES

FIG. 1A: is a diagrammatic representation showing the sequences of NCAM domain 1, the i-body scaffold of the present disclosure and CDR1 and CDR3 sequences of a specific RANK-L binder, ADR03. X and Y′ correspond to any amino acid respectively and n is any number between 10 and 20 inclusive.

FIG. 1B: is a CLUSTAL 0(1.2.4) multiple sequence alignment of RANKL binders.

FIG. 2: is a graphical representation showing Size Exclusion Chromatography purification of ADR03 and another i-body 117-lm7-FH.

FIG. 3: is a graphical representation of BIAcore binding data for ADR03 binding to RANK-L using surface plasmon resonance (SPR) showing an affinity of 13.2.:t. 3.9.

FIG. 4: The crystal structure of 21HS (human NCAM1, lg1 domain, PDB: 5AEA). A. Red and Purple colors highlight the inserted loops (CDR1 and CDR3, respectively). B. Predicted ADR03 structure in silica docked to human RANKL (PDB: 5BNQ). In the best-fit computational model, Red and Purple colors indicate the CDR1 and CDR3 loops which were shown to be involved in ADR03-RANKL interaction.

FIG. 5: Thermal stability of ADR03. Derivative curve displaying the thermal shift at different pHs, suggesting that ADR03 favours neutral or a more alkaline environment over a more acidic environment. Heatmap displaying the correlation between pH and the thermal stability of ADR03, where white colour represents a lower Tm and red colour indicates a higher Tm.

FIG. 6: Shows MTS assay showed no cytotoxicity of ADR3 on murine-isolated bone marrow cells (BMMs). The bar chart is presented as the mean±SD. (n=4).

FIG. 7: is a graphical representation showing ADR03 (7 μg/ml) inhibition of TRAP activity in mouse RAW264.7 cells that have been made TRAP positive by the addition of human RANK-L (hRANK-L) at a concentration of 60 ng/ml The i-body ADR03 demonstrated an IC50 in this assay of 3 nM. Line chart is represented as the mean. t. SD (n=4).

FIG. 8: the top panel (left to right) shows mouse RAVV264.7 cells before the addition of hRANK-L or ADR03, followed by the addition of hRANK-L (60 ng/ml) causing the cells to become TRAP positive. The bottom panel (left to right) shows mouse RAW264 cells with the addition of ADR03 (7 μg/ml), followed RAW264.7 cells treated with hRANK-L (60 ng/ml) to cause TRAP activity that is inhibited by ADR03 (7 μg/ml).

FIG. 9: is a graphical representation showing the inhibition of human RANK-L osteoclastogenesis in mouse RAW264.7 cells by hRANK-L RX i-body (ADR03) measured by a decrease in TRAP activity. hRANK-L CX (negative control i-body) and hRANK-L FF (negative control i-body) did not inhibit hRANK-L osteoclastogenesis or decrease TRAP activity, compared with the specific i-body for hRANKL ADR03 which inhibited osteoclastogenesis.

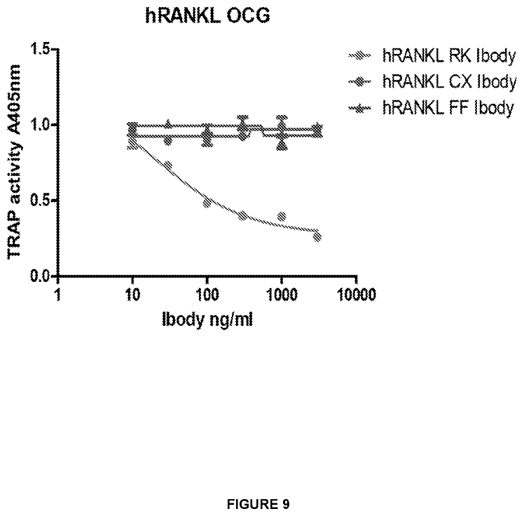

FIG. 10: is a graphical representation showing the inhibition of mouse RANK-L osteoclastogenesis in mouse RAW264.7 cells by mRANK-L RX i-body (ADR03) measured by a decrease in TRAP activity. mRANK-L CX (negative control i-body) and mRANK-L FF (negative control i-body) did not inhibit mRANK-L osteoclastogenesis or decrease TRAP activity, compared to the specific i-body for hRANKL ADR03 which inhibited osteoclastogenesis.

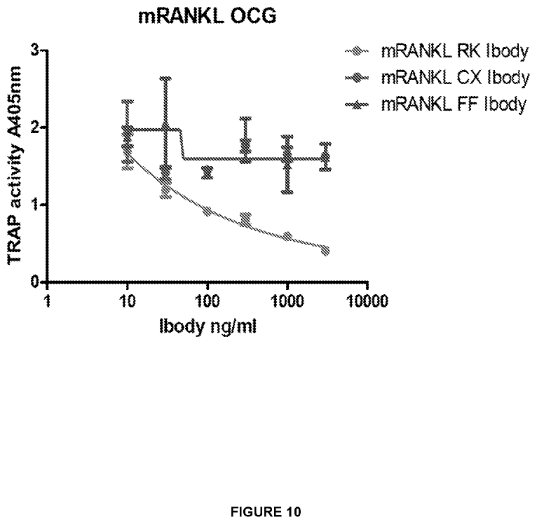

FIG. 11: i-body ADR03 inhibited RANKL-induced osteroclastogenesis. TRAcP staining (A) and podosome belt staining (B) of non-induced or RANKL-induced BMMs treated with or without 1 mM i-body ctrl, 0.5 mM ADR03 and 1 mM ADR03 respectively. Scale bar=200 mm (C). Quantification of TRAcP-positive cells in each group. (n=4) All data are presented as the mean±SD. ****p<0.0001 compared to the positive control. ####p<0.0001 compared between two groups of ADR3. (D). RAW264.7 cells stably transfected with an NFATc1 luciferase reporter construct were treated with RANKL alone or with i-body control, 0.5 mM and 1 mM ADR03 respectively. Cells were harvested to detect the luciferase activity. (n=4) The bar graph was constructed as the mean±SD. ****p<0.0001 compared to the positive control. ####p<0.0001 compared between two doses of ADR03.

FIG. 12: ADR03 suppressed hRANKL-induced bone resorption. A. Fresh BMMs were induced by 50 ng/ml RANKL to form TRAcP-positive osteoclasts on osteo assay surface plates. Representative resorption pits (n=3) are visualized in parallel with TRAcP staining (n=3). Scale bar=200 mm. B. Quantification of resorbed hydroxyapatite area per cell in each group. The bar graph is presented by the mean±SD. ***p<0.001 compared to the negative control.

FIG. 13: Osteoclastic markers (Calcr1, c-Fos, Nfatc1, Atpv0d2, Acp5, Ctsk and Mmp9) were downregulated by ADR03 treatment in a dose-dependent manner. The bar graphs are presented as mean.:t. SD (n=3) **p<0.01; ***p<0.001; ****p<0.0001 compared to the positive control. #p<0.05; ##p<0.01; ####p<0.0001 compared between the two doses of ADR03.

FIG. 14: RANKL-mediated downstream signaling was disrupted does-dependently by ADR03. A. Representative Western Blotting images showing the effect of ADR03 on RANKL-induced NFATc1, c-Fos, D2 and Ctsk at the protein level. B. The ratios of the densities of NFATc1, c-Fos, D2 and Ctsk bands relative to [3-actin bands were generated through ImageJ. C. Representative images displaying the protective effect of ADR03 on RANKL-regulated antioxidant enzymes HO-1 and Catalase. D. Protein expression levels were normalized to [3-actin and quantified through measuring grey values in ImageJ. All data are presented by the mean±SD. (n=3) *p<0.05; **p<0.01; ***p<0.001; ****p<0.0001 compared to the positive control. #p<0.05 compared between two doses of ADR03.

FIG. 15: ADR03 interrupts intracellular calcium mobility. A Ca²+ oscillation was measured by the Fluo-4 AM calcium indicator in the absence or presence with 50 ng/ml RANKL pre-mixed with or without i-body control, 0.5 mM and 1 mM ADR03 respectively for overnight before the fluorescence signal was captured every 2s for 3 minutes. B. The intensity of fluorescence and the percentage of oscillated cells in each group were quantified into bar charts presented as the mean±SD (n=3) **p<0.01; ***p<0.001 compared to the positive control. #p<0.05 compared between the two doses of ADR03.

FIG. 16: ADR03 enhanced the mobility of HUVECs. A. Representative images enlarged from Transwell assay. Scale bar=200 mm B. Crystal-violet stained cells were quantified via ImageJ. The bar chart is constructed as mean±SD. (n=3) **p<0.01 compared to Negative ctrl.

### KEY TO SEQUENCE LISTING

SEQ ID NO 1: amino acid sequence encoding Homo sapiens NCAM domain 1 also known as the i-body scaffold

SEQ ID NO: 2 amino acid sequence encoding i-body

SEQ ID NO: 3 amino acid sequence encoding Bos taurus NCAM domain 1

SEQ ID NO: 4 amino acid sequence encoding Mus musculus NCAM domain 1

SEQ ID NO: 5 amino acid sequence encoding Rat rattus NCAM domain 1

SEQ ID NO: 6 amino acid sequence encoding Gallus gallus NCAM domain 1

SEQ ID NO: 7 amino acid sequence encoding Xenopus laevis NCAM2 domain 1

SEQ ID NO: 8 amino acid sequence encoding Xenopus laevis NCAM1 domain 1

SEQ ID NO: 9 amino acid sequence encoding Homo sapiens NCAM2 domain 1

SEQ ID NO: 10 amino acid sequence encoding Mus musculus NCAM2 domain 1

SEQ ID NO: 11 amino acid sequence encoding ADR03

SEQ ID NO: 12 amino acid sequence encoding ADR03 CDR1

SEQ ID NO: 13 amino acid sequence encoding ADR03 CDR3

SEQ ID NO: 14 nucleotide sequence encoding ADR03

SEQ ID NO: 15 sequence of CDR1 of NCAM domain 1 SEQ ID NO: 16 sequence of CDR3 of NCAM domain 1

### DETAILED DESCRIPTION

### Selected Definitions

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The term “ADR3” and “ADR03” are to be used interchangeably herein.

As used herein, the term “binds” in reference to the interaction of a binding molecule with a target means that the interaction is dependent upon the presence of a particular structure (e.g., an antigenic determinant or epitope) on the target. For example, a binding molecule recognizes and binds to a specific protein structure rather than to proteins generally.

As used herein, the term “specifically binds” shall be taken to mean a binding molecule of the disclosure reacts or associates more frequently, more rapidly, with greater duration and/or with greater affinity with a particular target or cell expressing same than it does with alternative targets or cells. For example, a binding molecule that specifically binds to a target binds that target with greater affinity (e.g., 20 fold or 40 fold or 60 fold or 80 fold to 100 fold or 150 fold or 200 fold greater affinity), avidity, more readily, and/or with greater duration than it binds to other antigens, e.g., to other ligands commonly recognized by polyreactive natural antibodies (i.e., by naturally occurring antibodies known to bind a variety of antigens naturally found in humans). It is also understood by reading this definition that, for example, a binding molecule that specifically binds to a first target may or may not specifically bind to a second target. As such “specific binding” does not necessarily require exclusive binding or non-detectable binding of another target, this is meant by the term “selective binding”.

As used herein, reference to a “similar” level of binding will be understood to mean that a binding molecule binds to a target at a level within about 30% or 25% or 20% of the level at which it binds to another target. This term can also mean that one binding molecule binds to a target at a level within about 30% or 25% or 20% of the level at which another binding molecule binds to the same target.

As used herein, reference to “substantially the same level” of binding will be understood to mean that a binding molecule binds to a target at a level within about 15% or 10% or 5% of the level at which it binds to another target. This term can also mean that one binding molecule binds to a target at a level within about 5% or 4% or 3% of the level at which another binding molecule binds to the same target.

As used herein, the term “complementarity determining regions” (syn. CDRs; i.e., CDR1 and CDR3) refers to the amino acid residues within an immunoglopbulin superfamily domain, the presence of which are major contributors to specific antigen binding. The CDRs may also be referred to as “binding loops”.

### I-Body Scaffold and RANK-L Binding Molecules

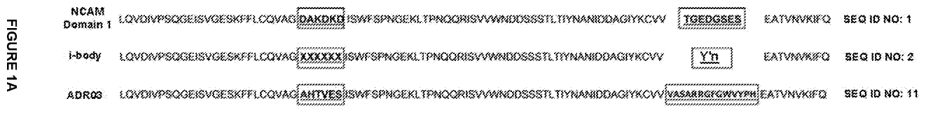

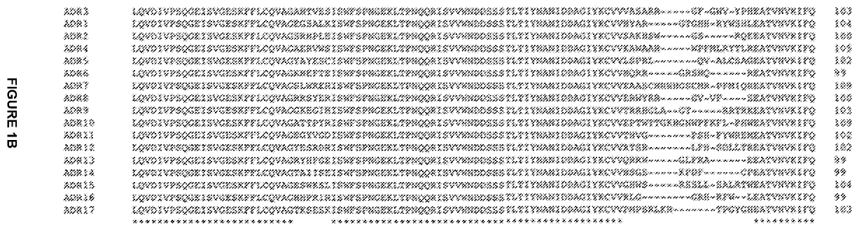

The present disclosure provides a polypeptide (or “i-body”) which comprises a scaffold with CDR1 and CDR3 regions. In one example the scaffold region comprises Domain 1 of NCAM as shown in SEQ ID NO:1 or a related domain sequence that has at least 45% identity thereto or at least 75% homology excluding CDR1 and CDR3 regions as highlighted. The sequence of one exemplary i-body scaffold is also shown FIG. 1A (where the CDR1 and CDR3 regions are highlighted).

NCAM is a glycoprotein of Immunoglobulin (lg) superfamily. The extracellular domain of NCAM consists of five immunoglobulin-like (lg) domains followed by two fibronectin type III (FNIII) domains.

Related domain sequences include SEQ ID NO's 3, 4, 5, 6 and 8 which show cow, mouse, rat, chicken and frog NCAM domain 1 sequences respectively and SEQ ID NO's 7, 9 and 10 which show frog, human and mouse NCAM 2 domain sequences respectively.

The sequence identity between these related domains is as follows:

| NCBI REF | Score | E value |
|---|---|---|
| P13591.3INCAM1_HUMAN RecName: | 200 | 5e−60 Identity (100%) |
| NCAM ID NO: 1 | | SEQ |
| P31836.1INCAM1_BOVIN RecName: | 197 | 6e−59 Identity (98%) |
| NCAM ID NO: 3 | | SEQ |
| P13595.3INCAM1_MOUSE RecName: | 195 | 2e−57 Identity (95%) |
| NCAM ID NO: 4 | | SEQ |
| P13596.1INCAM1_RAT RecName: | 192 | 3e−57 Identity (95%) |
| NCAM ID NO: 5 | | SEQ |
| P13590.3INCAM1_CHICK RecName: | 175 | 2e−50 Identity (89%) |
| NCAM ID NO: 6 | | SEQ |
| P36335.1INCA12_XENLA RecName: | 130 | 1e−34 Identity (68%) |
| NCAM ID NO: 7 | | SEQ |
| P16170.1INCA11_XENLA RecName: | 130 | 1e−34 Identity (68%) |
| NCAM ID NO: 8 | | SEQ |
| O15394.2INCAM2_HUMAN RecName: | 87.8 | 8e−20 Identity (51%) |
| NCAM ID NO: 9 | | SEQ |
| O35136.1INCAM2_MOUSE RecName: | 86.3 | 3e−19 Identity(47%) |
| NCAM ID NO: 10 | | SEQ |

The sequence homology between these related domains is as follows:

| | |
|---|---|
| spIP13591.3INCAM1_HUMAN | Sequence homology (100%) SEQ ID |
| RecName: NCAM | NO: 1 |
| spIP31836.1INCAM1_BOVIN | Sequence homology (100%) SEQ ID |
| RecName: NCAM | NO: 3 |
| spIP13595.3INCAM1_MOUSE | Sequence homology (99%) SEQ ID |
| RecName: NCAM | NO: 4 |
| sp1P13596.1 INCAM1_RAT | Sequence homology (99%) SEQ ID |
| RecName: NCAM | NO: 5 |
| sp1P13590.31NCAM1_CHICK | Sequence homology (98%) SEQ ID |
| RecName: NCAM | NO: 6 |
| splP36335.1INCA12_XENLA | Sequence homology (89%) SEQ ID |
| RecName: NCAM | NO: 7 |
| sp1P16170.1 INCA11_XENLA | Sequence homology (89%) SEQ ID |
| RecName: NCAM | NO: 8 |
| splO15394.21NCAM2_HUMAN | Sequence homology (74%) SEQ ID |
| RecName: NCAM | NO: 9 |
| splO35136.1 INCAM2_MOUSE | Sequence homology (74%) SEQ ID |
| RecName: NCAM | NO: 10 |

Domain 1 of NCAM has been produced as a recombinant polypeptide in a bacterial expression system (Frei et al. (1992) J. Cell Biol. 118:177-194).

The present invention describes introduced modifications in to an i-body scaffold in the CDR1 or CDR3 regions, and have shown that these modifications alter the binding properties of the domain (or “i-body”). In particular, the inventors have developed modified i-body amino acids and polypeptides which surprisingly are able to bind to RANK-L with high affinity and specificity and inhibit or reduce RANK-L induced osteoclastogenesis in in vitro models of osteoclast formation.

Accordingly the present disclosure provides a number of polypeptides which bind to RANK-L and comprises the i-body scaffold acid sequence, wherein the CDR1 or CDR3 region of the i-body scaffold have been modified and wherein the molecule binds to human or mouse RANK-L with an affinity of less than 200 nM.

In one embodiment the entire CDR1 or CDR3 regions of the scaffold are replaced with a randomised loop sequence.

For example, the CDR1 loop region of the scaffold may be replaced with a loop region having the sequence as shown in SEQ ID NO:12 or a sequence having 90% identity thereto.

In another example, the CDR3 loop region of the scaffold may be replaced with a loop region having the sequence as shown in SEQ ID NO:13 or a sequence having 90% identity thereto.

In one example the polypeptide comprises a sequence that has at least 80% identity, at least 90% identity, or at least 95% identity, or at least 97% identity, or at least 98% identity, or at least 99% identity to identity to SEQ ID NO: 11.

In one example the polypeptide comprises or consists of SEQ ID NO:11.

The present disclosure also provides a nucleic acid molecule encoding a polypeptide described herein.

In one example the nucleic acid molecule comprises a sequence that has at least 80% identity, at least 90% identity, or at least 95% identity, or at least 97% identity, or at least 98% identity, or at least 99% identity to identity to any one of SEQ ID NO 14.

The % identity of a polypeptide or polynucleotide is determined by GAP (Needleman and Wunsch. 1970) analysis (GCG program) with a gap creation penalty=5, and a gap extension penalty=0.3. The query sequence is at least 50 residues in length, and the GAP analysis aligns the two sequences over a region of at least 50 residues. For example, the query sequence is at least 100 residues in length and the GAP analysis aligns the two sequences over a region of at least 100 residues. In one example, the two sequences are aligned over their entire length.

For purposes of the present disclosure, alignments of sequences and calculation of homology scores are done using a Needleman-Wunsch alignment (i.e. global alignment), useful for both protein and DNA alignments. The default scoring matrices BLOSUM50 and the identity matrix are used for protein and DNA alignments respectively. The penalty for the first residue in a gap is −12 for proteins and −16 for DNA, white the penalty for additional residues in a gap is −2 for proteins and −4 for DNA. Alignment is from the FASTA package version v20u6 (W. R. Pearson and D. J. Lipman (1988), “Improved Tools for Biological Sequence Analysis”, PNAS 85:2444-2448, and W. R. Pearson (1990) “Rapid and Sensitive Sequence Comparison with FASTP and FASTA”, Methods in Enzymology, 183:63-98).

The present disclosure contemplates variant forms of binding protein of the disclosure. For example, such a variant binding protein comprises one or more conservative amino acid substitutions compared to a sequence set forth herein. In some examples, the binding protein comprises 10 or fewer, e.g., 9 or 8 or 7 or 6 or 5 or 4 or 3 or 2 or 1 conservative amino acid substitutions. A “conservative amino acid substitution” is one in which the amino acid residue is replaced with an amino acid residue having a similar side chain and/or hydropathicity and/or hydrophilicity.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, praline, phenylalanine, methionine, tryptophan), /3-branched side chains (e.g., threonine, valine, isoleucine) and aromatic side chains (e.g., tyrosine, phenylalanine, tryptophan, histidine). Hydropathic indices are described, for example in Kyte and Doolittle (1982) and hydrophylic indices are described in, e.g., U.S. Pat. No. 4,554,101.

The present disclosure also contemplates non-conservative amino acid changes. For example, of particular interest are substitutions of charged amino acids with another charged amino acid and with neutral or positively charged amino acids. In some examples, the binding protein comprises 10 or fewer, e.g., 9 or 8 or 7 or 6 or 5 or 4 or 3 or 2 or 1 non-conservative amino acid substitutions.

A variant form of a RANK-L binding protein described herein according to any example retains the ability to bind to RANK-L. Methods for determining specific binding to RANK-L are described herein.

### Affinity Maturation

In a further example, an existing binding protein of the disclosure is affinity matured to produce an i-body capable of binding to RANK-L with increased affinity, specificity or activity or to produce an i-body with increased expression or solubility. For example, the sequence encoding the binding protein is mutated such that one or more amino acid substitutions is introduced. The resulting variant binding protein is then screened for binding to RANK-L, e.g., in a competitive assay, screened for increase in specificity with various compounds (TNF-a, TRAIL or CD40L), or screened for increase in expression or increase in solubility or screened via affinity assays as described below for increases in affinity.

There are several protocols for affinity maturation of polypeptides and proteins. These include DNA shuffling (Stemmer Proc Natl Acad Sc! USA. (1994); 91(22):10747-10751), error-prone PCR (Hawkins et al. (1992) J. Mol. Biol., 226; 889-896 and Henderson et al. (2007) Structure 15:1452-66) and bacterial mutator cells (Irving et al. (1996) Immunotechnology 2:127-43.3) that randomise the whole scaffold, as well as more targeted methods such as doped oligonucleotide mutagenesis (Hermes et al. (1989) Gene, 84; 143-1514). Ribosome display coupled with error-prone RNA dependent RNA polymerase from Qbeta bacteriophage has also been used to affinity mature single domains, binding proteins and polypeptides (Kopsidas et al, (2006) Immunology Letters 107 163-168).

The binding proteins according to the disclosure may be soluble secreted proteins or may be presented as a fusion protein on the surface of a cell, or particle (e.g., a phage or other virus, a ribosome or a spore). Exemplary phage display methods are described, for example, in U.S. Pat. Nos. 5,821,047; 6,248,516 and 6,190,908. Phage display particles produced using these methods are then screened to identify a displayed binding protein having a conformation sufficient for binding to RANK-L.

### Protein Production

In one example, a polypeptide of the disclosure is produced by culturing a cell line, e.g., an E. coli cell line under conditions sufficient to produce the protein, e.g., as described herein and/or as is known in the art.

### Recombinant Expression

In the case of a recombinant protein, nucleic acid encoding same is placed into one or more expression construct, e.g., expression vector(s), which is/are then transfected into host cells, such as cells that can produce a disulphide bridge or bond, such as bacterial cells including E. coli cells, yeast cells, insect cells, or mammalian cells. Exemplary mammalian cells include simian COS cells, Chinese Hamster Ovary (CHO) cells, or myeloma cells that do not otherwise produce immunoglobulin protein. Exemplary bacterial cells include BL21(DE3), BL21(DE3)-plysS, Tuner, Tuner plysS, Origami, Origami B, Origami B plysS, Rosetta, AD494, HMS174 which are all available form Novagen.

Molecular cloning techniques to achieve these ends are known in the art and described, for example in Ausubel or Sambrook. A wide variety of cloning and in vitro amplification methods are suitable for the construction of recombinant nucleic acids.

Following isolation, the nucleic acid encoding a protein of the disclosure is inserted into an expression construct or replicable vector for further cloning (amplification of the DNA) or for expression in a cell-free system or in cells. For example, the nucleic acid is operably linked to a promoter,

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Exemplary promoters can contain additional copies of one or more specific regulatory elements to further enhance expression and/or alter the spatial expression and/or temporal expression of said nucleic acid.

As used herein, the term “operably linked to” means positioning a promoter relative to a nucleic acid such that expression of the nucleic acid is controlled by the promoter.

Cell free expression systems are also contemplated by the present disclosure. For example, a nucleic acid encoding a RANK-L binding polypeptide is operably linked to a suitable promoter, e.g., a T7 or TS promoter, and the resulting expression construct exposed to conditions sufficient for transcription and translation. Typical expression vectors for in vitro expression or cell-free expression have been described and include, but are not limited to the TNT T7 and TNT T3 systems (Promega), the pEXP1-DEST and pEXP2-DEST vectors (Invitrogen).

Many vectors for expression in cells are available. The vector components generally include, but are not limited to, one or more of the following: a signal sequence, a sequence encoding a binding protein of the present disclosure (e.g., derived from the information provided herein), an enhancer element, a promoter, and a transcription termination sequence. The skilled artisan will be aware of suitable sequences for expression of a protein. For example, exemplary signal sequences include prokaryotic secretion signals (e.g., DsbA, pelB, alkaline phosphatase, penicillinase, lpp, or heat-stable enterotoxin II), yeast secretion signals (e.g., invertase leader, a factor leader, or acid phosphatase leader) or mammalian secretion signals (e.g., herpes simplex gD signal).

Exemplary leader peptides include those active in prokaryotes (such as PelB, OmpA, PIii, DsbA, TorT, TolB, phoA promoter, [3-lactamase and lactose promoter systems, alkaline phosphatase, a tryptophan (trp) promoter system, and hybrid promoters such as the tac promoter).

Suitable bacterial promoters include the E. coli lacl and lacZ promoters, the T3 and T7, TS promoters, the gpt promoter, the lambda PR and PL promoters and the trp promoter. Eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

Exemplary promoters active in mammalian cells include cytomegalovirus immediate early promoter (CMV-IE), human elongation factor 1-a promoter (EF1), small nuclear RNA promoters (Ula and Ulb), a-myosin heavy chain promoter, Simian virus 40 promoter (SV40), Rous sarcoma virus promoter (RSV), Adenovirus major late promoter, [3-actin promoter; hybrid regulatory element comprising a CMV enhancer/[3-actin promoter or an immunoglobulin promoter or active fragment thereof. Examples of useful mammalian host cell lines are monkey kidney CV1 line transformed by SV40 (COS-7, AUSTRALIAN CELL BANK CRL 1651); human embryonic kidney line (293 or 293 cells subcloned for growth in suspension culture; baby hamster kidney cells (BHK, AUSTRALIAN CELL BANK CCL 10); or Chinese hamster ovary cells (CHO).

Typical promoters suitable for expression in yeast cells such as for example a yeast cell selected from the group comprising Pichia pastoris, Saccharomyces cerevisiae and S. pombe, include, but are not limited to, the ADH1 promoter, the GAL1 promoter, the GAL4 promoter, the CUP1 promoter, the PH05 promoter, the nmt promoter, the RPR1 promoter, or the TEF1 promoter.

Means for introducing the isolated nucleic acid molecule or a gene construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation, viral transduction (e.g., using a lentivirus) and microparticle bombardment such as by using DNA-coated tungsten or gold particles (Agracetus Inc., WI, USA) amongst others.

In some cases it is useful to express a protein or peptide in insoluble form, particularly when the peptide of interest is rather short, normally soluble, and/or subject to proteolytic degradation within the host cell. Production of the protein in insoluble form both facilitates simple recovery and protects the peptide from the undesirable proteolytic degradation. One means to produce the peptide in insoluble form is to recombinantly produce the peptide as part of an insoluble fusion protein by including in the fusion construct at least one peptide tag (i.e., an inclusion body tag) that induces inclusion body formation. Typically, the fusion protein is designed to include at least one cleavable peptide linker so that the peptide of interest can be subsequently recovered from the fusion protein. The fusion protein may be designed to include a plurality of inclusion body tags, cleavable peptide linkers, and regions encoding the peptide of interest.

Fusion proteins comprising a peptide tag that facilitate the expression of insoluble proteins are well known in the art. Typically, the tag portion of the chimeric or fusion protein is large, increasing the likelihood that the fusion protein will be insoluble. Example of large peptide tides typically used include, but are not limited to chloramphenicol acetyltransferase (Dykes et al., Eur. J. Biochem., 174:411 (1988), .beta.-galactosidase (Schellenberger et al., Int. J. Peptide Protein Res., 41:326 (1993); Shen et al., Proc. Nat. Acad. Sci. USA 281:4627 (1984); and Kempe et al., Gene, 39:239 (1985)), glutathione-S-transferase (Ray et al., Bio/Technology, 11:64 (1993) and Hancock et al. (WO94/04688)), the N-terminus of L-ribulokinase (U.S. Pat. No. 5,206,154 and Lai et al., Antimicrob. Agents & Chemo., 37:1614 (1993), bacteriophage T4 gp55 protein (Gramm et al., Bio/Technology, 12:1017 (1994), bacterial ketosteroid isomerase protein (Kuliopulos et al., J. Am. Chem. Soc. 116:4599 (1994), ubiquitin (Pilon et al., Biotechnol. Prag., 13:374-79 (1997), bovine prochymosin (Naught et al., Biotechnol. Bioengineer. 57:55-61 (1998), and bactericidal/permeability-increasing protein (“BPI”; Better, M. D. and Gavit, PD., U.S. Pat. No. 6,242,219). The art is replete with specific examples of this technology, see for example U.S. Pat. No. 6,613,548, describing fusion protein of proteinaceous tag and a soluble protein and subsequent purification from cell lysate; U.S. Pat. No. 6,037,145, teaching a tag that protects the expressed chimeric protein from a specific protease; U.S. Pat. No. 5,648,244, teaching the synthesis of a fusion protein having a tag and a cleavable linker for facile purification of the desired protein; and U.S. Pat. Nos. 5,215,896; 5,302,526; 5,330,902; and US 2005221444, describing fusion tags containing amino acid compositions specifically designed to increase insolubility of the chimeric protein or peptide.

Shorter inclusion body tags have recently been developed from the Zea mays zein protein (co-owned U.S. patent application Ser. No. 11/641,936), the Daucus carota cystatin (co-owned U.S. patent application Ser. No. 11/641,273), and an amyloid-like hypothetical protein from Caenorhabditis elegans (co-owned U.S. patent application Ser. No. 11/516,362; each hereby incorporated by reference in their entirety.) The use of short inclusion body tags increases the yield of the target peptide produced within the recombinant host cell.

The host cells used to produce the binding protein of this disclosure may be cultured in a variety of media, depending on the cell type used. Commercially available media such as Ham's FIO (Sigma), Minimal Essential Medium ((MEM), (Sigma), RPMl-1640 (Sigma), and Dulbecco's Modified Eagle's Medium ((DMEM), Sigma) are suitable for culturing mammalian cells. Media for culturing other cell types discussed herein are known in the art.

### Isolation of Proteins

A binding protein of the present disclosure can be isolated or purified.

Methods for purifying a binding molecule of the disclosure are known in the art and/or described herein.

When using recombinant techniques, the binding protein of the disclosure can be produced intracellularly, in the periplasmic space, or directly secreted into the medium. If the protein is produced intracellularly, as a first step, the particulate debris, either host cells or lysed fragments, is removed, for example, by centrifugation or ultrafiltration. Where the protein is secreted into the medium, supernatants from such expression systems can be first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The protein prepared from the cells can be purified using, for example, ion exchange, hydroxyapatite chromatography, hydrophobic interaction chromatography, gel electrophoresis, dialysis, affinity chromatography (e.g., protein A affinity chromatography or protein G chromatography), heat, or any combination of the foregoing. These methods are known in the art and described, for example in WO99/57134 or Zola (1997).

The skilled artisan will also be aware that a binding protein of the disclosure can be modified to include a tag to facilitate purification or detection, e.g., a poly-histidine tag, e.g., a hexa-histidine tag, or a influenza virus hemagglutinin (HA) tag, or a Simian Virus 5 (VS) tag, or a FLAG tag, or a glutathione S-transferase (GST) tag. For example, the tag is a hexa-his tag.

The resulting protein is then purified using methods known in the art, such as, affinity purification. For example, a protein comprising a hexa-his tag is purified by contacting a sample comprising the protein with nickel-nitrilotriacetic acid (Ni-NTA) that specifically binds a hexa-his tag immobilized on a solid or semi-solid support, washing the sample to remove unbound protein, and subsequently eluting the bound protein. Alternatively, or in addition a ligand or antibody that binds to a tag is used in an affinity purification method.

### Conjugates

The present disclosure also provides conjugates of RANK-L-binding molecules described herein according to any example. Examples of compounds to which a binding molecule can be conjugated are selected from the group consisting of a radioisotope, a detectable label, a therapeutic compound, a colloid, a toxin, a nucleic acid, a peptide, a protein, a compound that increases the half life of the protein in a subject and mixtures thereof. Exemplary therapeutic agents include, but are not limited to an anti-angiogenic agent, an anti-neovascularization and/or other vascularization agent, an anti-proliferative agent, a pro-apoptotic agent, a chemotherapeutic agent or a therapeutic nucleic acid.

A toxin includes any agent that is detrimental to (e.g., kills) cells. For a description of these classes of drugs which are known in the art, and their mechanisms of action, see Goodman et al., (1990). Additional techniques relevant to the preparation of i-body-immunotoxin conjugates are provided in for instance in U.S. Pat. No. 5,194,594. Exemplary toxins include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin and the tricothecenes. See, for example, WO93/21232.

Suitable chemotherapeutic agents for forming immunoconjugates of the present disclosure include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1 de-hydrotestosterone, glucocorticoids, procaine, tetracaine, lidocaine, propranolol, and puromycin, antimetabolites (such as methotrexate, 6-mercaptopurine, 6 thioguanine, cytarabine, fludarabin, 5-fluorouracil, decarbazine, hydroxyurea, asparaginase, gemcitabine, cladribine), alkylating agents (such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin), antibiotics (such as dactinomycin (formerly actinomycin), bleomycin, daunorubicin (formerly daunomycin), doxorubicin, idarubicin, mithramycin, mitomycin, mitoxantrone, plicamycin, anthramycin (AMC)).

In one example, an RANK-L-binding polypeptide as described herein is conjugated or linked to another protein, including another RANK-L-binding molecule of the disclosure or a protein comprising a CDR1 and/or CDR3 region as described herein. A RANK-L-binding polypeptide as described herein may also be conjugated to another binding molecule which targets, for example, a tumour antigen, or a target that has the potential to redirect and activate any circulating T cells against tumors (for example CD3), or a target that is notably expressed on monocytes and macrophages and upregulated upon activation on neutrophils (for example CD64) or a target that is expressed on the surface of natural killer cells, neutrophil polymorphonuclear leukocytes, monocytes and macrophages. In one example the binding polypeptide is a low affinity binder of IgG (for example CD16) or a target that is constitutively expressed primarily on neutrophils, monocytes, macrophages and eosinophils (for example CD89). other proteins or conjugation partners are not excluded. Additional proteins will be apparent to the skilled artisan and include, for example, an immunomodulator or a half-life extending protein or a peptide or other protein that binds to serum albumin amongst others.

Exemplary serum albumin binding peptides or protein are described in US20060228364 or US20080260757.

In one example a polypeptide of the present disclosure is conjugated to an XTEN polypeptide as described in Schellenberger et al (2009) nature biotechnology 27(12):1186-1192.

In one example a polypeptide of the present disclosure is conjugated to an Fe region of an immunoglobulin as described, for example, in Peters et al (2010), Blood Vol. 115 no. 10 2057-2064, Kim et al, (2009) BMB Rep. 42:212-216 and Nagashima et al (2011) J Biochem. 149: 337-346.

A variety of radionuclides are available for the production of radioconjugated proteins. Examples include, but are not limited to, low energy radioactive nuclei (e.g., suitable for diagnostic purposes), such as 13C, 15N, 2H, 1251, 1231, 99Tc, 43K, 52Fe, 67Ga, 68Ga, 111In and the like. For example, the radionuclide is a gamma, photon, or positron-emitting radionuclide with a half-life suitable to permit activity or detection after the elapsed time between administration and localization to the imaging site. The present disclosure also encompasses high energy radioactive nuclei (e.g., for therapeutic purposes), such as 1251, 1311, 1231, 111In, 105Rh, 153Sm, 67Cu, 67Ga, 166Ho, 177Lu, 186Re and 188Re. These isotopes typically produce high energy a- or [3-particles which have a short path length. Such radionuclides kill cells to which they are in close proximity, for example neoplastic cells to which the conjugate has attached or has entered. They have little or no effect on non-localized cells and are essentially non-immunogenic. Alternatively, high-energy isotopes may be generated by thermal irradiation of an otherwise stable isotope, for example as in boron neutron-capture therapy (Guan et al., 1998). other isotopes which may be suitable are described in Carter. (2001) Nature Reviews Cancer 1, 118-129, Goldmacher et al. (2011) Therapeutic Delivery 2; 397-416, Payne (2003) Cancer Cell 3, 207-212, Schrama et al, (2006) Nature Rev. Drug Discov. 5, 147-159, Reichert et al. (2007) Nature Reviews Drug Discovery 6; 349-356.

In another example, the protein is conjugated to a “receptor” (such as streptavidin) for utilization in cell pretargeting wherein the conjugate is administered to the patient, followed by removal of unbound conjugate from the circulation using a clearing agent and then administration of a “ligand” (e.g., avidin) that is conjugated to a therapeutic agent (e.g., a radionucleotide).

The RANK-L-binding proteins of the present disclosure can be modified to contain additional nonproteinaceous moieties that are known in the art and readily available. For example, the moieties suitable for derivatization of the protein are physiologically acceptable polymer, e.g., a water soluble polymer. Such polymers are useful for increasing stability and/or reducing clearance (e.g., by the kidney) and/or for reducing immunogenicity of a RANK-L-binding protein of the disclosure. Non-limiting examples of water soluble polymers include, but are not limited to, polyethylene glycol (PEG), polyvinyl alcohol (PVA), or propropylene glycol (PPG).

In one example, a RANK-L-binding protein as described herein according to any example comprises one or more detectable markers to facilitate detection and/or isolation. For example, the compound comprises a fluorescent label such as, for example, fluorescein (FITC), 5,6-carboxymethyl fluorescein, Texas red, nitrobenz-2-oxa-I,3-diazol-4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7, fluorescein (5-carboxyfluorescein-N-hydroxysuccinimide ester), rhodamine (5,6-tetramethyl rhodamine). The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm).

Alternatively, or in addition, the RANK-L-binding protein as described herein according to any example is labelled with, for example, a fluorescent semiconductor nanocrystal (as described, for example, in U.S. Pat. No. 6,306,610).

Alternatively, or in addition, the RANK-L-binding protein is labelled with, for example, a magnetic or paramagnetic compound, such as, iron, steel, nickel, cobalt, rare earth materials, neodymium-iron-boron, ferrous-chromium-cobalt, nickel-ferrous, cobalt-platinum, or strontium ferrite.

### Immobilized Proteins

In one example a binding protein of the disclosure is immobilized on a solid or semi-solid matrix. The term “immobilization” is to be understood to involve various methods and techniques to fix proteins onto specific matrices, e.g. as described in WO99/56126 or WO02/26292. For example, immobilization can serve to stabilize the proteins so that its activity is not reduced or adversely modified by biological, chemical or physical exposure, especially during storage or in single-batch use.

In the meaning of the disclosure, three basic methods can be used for immobilization:

Various methods for immobilizing a protein on a matrix are known in the art and include crosslinking, binding to a carrier, retention within a semi-permeable matrix.

Exemplary matrices include porous gels, aluminium oxide, bentonite, agarose, starch, nylon or polyacrylamide.

### Assaying Activity of a Binding Molecules of the Disclosure

### Binding Assays

One form of such an assay is an antigen binding assay, e.g., as described in Scopes In: Protein purification: principles and practice, Third Edition, Springer Verlag, 1994. Such a method generally involves labelling the binding protein and contacting it with immobilized target or a fragment thereof, e.g., human RANK-L. Following washing to remove non-specific bound protein, the amount of label and, as a consequence, bound protein is detected. Of course, the binding protein can be immobilized and the target labelled. Panning-type assays, e.g., as described or exemplified herein can also be used.

### Affinity Assays

Optionally, the dissociation constant (Kd) or association constant (Ka) or binding constant (Ko, i.e., Ka/Kd) of a binding molecule for RANK-L is determined. These constants for a binding protein may be measured by a radiolabeled or fluorescently-labelled RANK-L binding assay. This assay equilibrates the binding protein with a minimal concentration of labelled RANK-L in the presence of a titration series of unlabelled RANK-L. Following washing to remove unbound RANK-L, the amount of label is determined. According to another example the constants are measured by using surface plasmon resonance assays, e.g., using BIAcore surface plasmon resonance (BIAcore, Inc., Piscataway, NJ) with immobilized RANK-Lor a region thereof.

### Osteoclast Assays

Bone homeostasis depends on balanced bone deposition and bone resorption, which are mediated by osteoblasts and osteoclasts, respectively. The process of bone turnover requires the coordination of these cells. Changes in the ability of either cell type to perform its function results in pathological conditions such as osteoporosis and tumor-induced bone loss (osteolysis). The number of osteoclasts present at the site of bone remodelling as well as the activity of those osteoclasts the control amount of bone resorbed. Therefore, factors affecting overall numbers of osteoclasts and osteoclast activation are key to regulating bone loss. Osteoclast numbers are in part controlled by osteoclast differentiation from bone marrow precursors of the monocyte/macrophage lineage. Differentiation of these hematopoietic precursors into osteoclasts is supported by RANK-L. Mechanistic studies to elucidate the factors influencing bone metabolism can involve in vitro studies of osteoclast differentiation, activation or survival. It will be appreciated that any of a number of in vitro assays can be used to assess the activity of the binding molecules of the present disclosure.

Tartarate Resistant Acid Phosphatase (TRAP) is a specific and sensitive indicator of bone resorption and contributes to the processing of primary bone matrix degradation products. When RAW264.7 cells are treated with RANK-Land colony stimulating factor they generate osteoclasts and which can be assessed by their TRAP activity (Quinn et al., Endocrinology, (1998) 139:4424-4427.

RAW cells can be purchased from American Type Culture Collection (Manassass, VA) and maintained, for example, in high glucose DMEM containing 10% fetal bovine serum and antibiotics. The cells may be sub-cultured bi-weekly to a maximum of 10-12 passages. For osteoclast differentiation experiments, RAW cells may be seeded, for example, in 96-well plates at a density of 10⁴ cells/well and allowed to plate for 24 h. Differentiation can be induced in high glucose DMEM and fetal calf serum and 100 ng/ml RANK-L. The plates may be re-fed on day 3 and osteoclasts are likely to be visible by day 4. Typically, the cells are then stained for TRAP on day 4 or 5.

Similar TRAP assays can be used with CD14+ monocytes as described in Duplomb et al., Endocrinology 149(7):3688-3697 (2008) and Costa-Rodriguez et al., Cell Prolif. 44(5):410-419 (2011).

Binding molecules of the present disclosure can also be evaluated for the ability to inhibit bone resorption in vitro is the bone slice/dentine disc assay as described in Boyd et al. (1984) British Dental Journal 156:216-220, Schilling et al. (2004) Biomaterials 25: 3963-3972 and Susa et al. (2004) Journal of Translational Medicine, 2:6 in which osteoclasts are seeded onto resorbable substrates and the excavation of resorption lacunae is measured.

### In Vivo Therapeutic Efficacy Assays

Animal models can be used in order to confirm the in vivo biological activity of binding molecules of the present invention. A non-human mammal having abnormal bone metabolism can be used, and a mouse, rat, hamster or cynomolgus monkey model is preferred.

Examples of animal models having abnormal bone metabolism include an animal having the ovary removed, an animal having the testicle removed, a cancer-bearing animal having tumor cells implanted under the skin, into the skin, left ventricle, bone marrow, vein, abdominal cavity or the like, an animal having a sciatic nerve removed, an animal model of adjuvant arthritis, an animal model of collagen-induced arthritis, an animal model of glucocorticoid-induced osteoporosis, a senescence-accelerated mouse (SAM P6 mouse, Matsushita et al., Am. J. Pathol. 125, 276-283 (1986)), an animal having the thyroid/parathyroid removed, an animal receiving a continuous infusion of a parathyroid hormone-related peptide (PTHrP), an osteoclastogenesis inhibitory factor (OCIF) knockout mouse (Mizuno et al., Biochem. Biophys. Res. Commun., (1998) 247, 610-615), an animal with the administration of soluble RANK-Lor the like can be used.

A binding molecule selected by screening can be administered to any of the above-mentioned animal models, and the therapeutic and/or preventive effect of the binding molecule on abnormal bone metabolism can be evaluated by measuring, for example, the number of mature osteoclasts in a bone tissue, bone density, bone strength or bone morphology, bone metabolism parameters (CTx, NTx, etc.) in blood and urine or parameters that vary due to abnormal bone metabolism such as blood calcium levels.

In one example, the biological activity of binding molecules of the present disclosure can be assessed by determining whether they are capable of blocking human RANK-L using a mouse model which has implanted slow release pellets containing human RANK-L, and a mouse expressing human RANK-L in place of the native murine protein. An example of this type of mouse model is described in Hofbauer et al., 2009; Arthritis and Rhematism 60(5): 1427-1437.

In another example, the biological activity of binding molecules of the present disclosure can be assessed by using an overiectomized cynomolgus monkey model as described in Kosternuik et al., (2011) Bone 49:151-161 and Ominsky et al., (2011) Bone 49:162-173 or an overiectomized mouse as described in Yamane et al, Bone (2009) 44: 1055-1062 or an overiectomized rat model as described in Jee et al, J Musculoskel Neuron Interact 2001; 1(3):193-207, Barlet et al (1994) Nutr Rev 34:221-236, Aerssens et al (1998) Endocrinology 139: 663-670, Wronski et al (1985) Calcif Tissue Int 37:324-328, Wronski et al (1991) Cells and Materials Suppl 1:69-74, Frost et al (1992) Bone Miner 18:227-236, Kalu (1991) Bone Miner 15:175-192, Dempster et al (1995), Bone 16:157-161. Various other animal models are detailed in Turner (2001), European Cells and Materials Vol 1: 66-81.

In another example, the ability of binding molecules of the present disclosure to inhibit skeletal tumor progression can be assessed by using a mouse model of breast cancer bone metastasis as described in Canon et al., Clin. Exp. Metastasis (2008) 25:119-129.

In another example, the ability of binding molecules of the present disclosure to inhibit skeletal tumor progression can be assessed by using a mouse model of prostate cancer bone metastasis as described in Armstrong et al, Prostate (2008) 68:920104, Miller et al, Mol Cancer Ther (2008), 7:2160-2169, Whang, P. G. et al J. Orthop. Res. 23, 1475-1483 (2005), Quinn, J. E. et al. Prostate Cancer Prostatic Dis. 8, 253-259 (2005), and Miller, R. E. et al. Mol. Cancer Ther. 7, 2160-2169 (2008).

### Diagnostic/Prognostic Assays

It will be apparent from the description herein that the present disclosure provides various methods for diagnosing/prognosing conditions associated with RANK-L expression.

One example of the disclosure detects the presence of RANK-L or a cell expressing same. The amount, level or presence of a protein or cell is determined using any of a variety of techniques known to the skilled artisan such as, for example, a technique selected from the group consisting of flow cytometry, immunohistochemistry, immunofluorescence, an immunoblot, a Western blot, a dot blot, an enzyme linked immunosorbent assay (ELISA), radioimmunoassay (RIA), enzyme immunoassay, fluorescence resonance energy transfer (FRET), matrix-assisted laser desorption/ionization time of flight (MALDI-TOF), electrospray ionization (ESI), mass spectrometry (including tandem mass spectrometry, e.g. LC MS/MS), biosensor technology, evanescent fiber-optics technology or protein chip technology.

In one example the assay used to determine the amount or level of a protein is a semi-quantitative assay.

In another example the assay used to determine the amount or level of a protein is a quantitative assay.

For example, the protein is detected with an immunoassay, e.g., using an assay selected from the group consisting of, immunohistochemistry, immunofluorescence, enzyme linked immunosorbent assay (ELISA), fluorescence linked immunosorbent assay (FLISA), Western blotting, radioimmunoassay (RIA), a biosensor assay, a protein chip assay and an immunostaining assay (e.g. immunofluorescence).

Standard solid-phase ELISA or FLISA formats are particularly useful in determining the concentration of a protein from a variety of samples.

In one form, an ELISA or FLISA comprises of immobilizing an RANK-L-binding protein of the disclosure or a protein that binds to a different epitope of RANK-L on a solid matrix, such as, for example, a membrane, a polystyrene or polycarbonate microwell, a polystyrene or polycarbonate dipstick or a glass support. A sample is then brought into physical relation with the immobilized protein, RANK-Lis bound or ‘captured’. The bound RANK-Lis then detected using a second labeled compound that binds to a different epitope of RANK-L (e.g., the RANK-L-binding protein of the disclosure). Alternatively, a third labeled antibody can be used that binds the second (detecting) antibody.

It will be apparent to the skilled person that the assay formats described herein are amenable to high throughput formats, such as, for example automation of screening processes or a microarray format. Furthermore, variations of the above-described assay will be apparent to those skilled in the art, such as, for example, a competitive ELISA.

In an alternative example, a polypeptide is detected within or on a cell, using methods known in the art, such as, for example, immunohistochemistry or immunofluorescence. Methods using immunofluorescence are exemplary, as they are quantitative or at least semi-quantitative. Methods of quantitating the degree of fluorescence of a stained cell are known in the art and described, for example, in Cuello, 1984.

Biosensor devices generally employ an electrode surface in combination with current or impedance measuring elements to be integrated into a device in combination with the assay substrate (such as that described in U.S. Pat. No. 5,567,301). A RANK-L-binding protein of the disclosure is incorporated onto the surface of a biosensor device and a biological sample contacted to said device. A change in the detected current or impedance by the biosensor device indicates protein binding to said RANK-L-binding protein. Some forms of biosensors known in the art also rely on surface plasmon resonance to detect protein interactions, whereby a change in the surface plasmon resonance surface of reflection is indicative of a protein binding to a ligand or antibody (U.S. Pat. Nos. 5,485,277 and 5,492,840).

Biosensors are of particular use in high throughput analysis due to the ease of adapting such systems to micro- or nano-scales. Furthermore, such systems are conveniently adapted to incorporate several detection reagents, allowing for multiplexing of diagnostic reagents in a single biosensor unit. This permits the simultaneous detection of several proteins or peptides in a small amount of body fluids.

### Imaging Methods

As will be apparent to the skilled artisan from the foregoing, the present disclosure also contemplates imaging methods using an RANK-L-binding protein of the disclosure. For imaging, an RANK-L-binding protein is generally conjugated to a detectable label, which can be any molecule or agent that can emit a signal that is detectable by imaging. However, a secondary labeled compound that specifically binds to an RANK-L-binding protein of the disclosure may also be used. Exemplary detectable labels include a protein, a radioisotope, a fluorophore, a visible light emitting fluorophore, infrared light emitting fluorophore, a metal, a ferromagnetic substance, an electromagnetic emitting substance a substance with a specific magnetic resonance (MR) spectroscopic signature, an X-ray absorbing or reflecting substance, or a sound altering substance.

The RANK-L-binding protein of the disclosure (and, if used the labeled secondary compound) can be administered either systemically or locally to an organ, or tissue (or tumor, in the case of a cancer) to be imaged, prior to the imaging procedure. Generally, the RANK-L-binding protein is administered in doses effective to achieve the desired optical image of a tumor, tissue, or organ. Such doses may vary widely, depending upon the particular RANK-L-binding protein employed, condition to be imaged, tissue, or organ subjected to the imaging procedure, the imaging equipment being used, and the like.

In some examples of the disclosure, the RANK-L-binding protein is used as in vivo optical imaging agents of tissues and organs in various biomedical applications including, but not limited to, imaging of tumours, tomographic imaging of organs, monitoring of organ functions, coronary angiography, fluorescence endoscopy, laser guided surgery, photoacoustic and sonofluorescence methods, and the like.

Examples of imaging methods include magnetic resonance imaging (MRI), MR

spectroscopy, radiography, computerized tomography (CT), ultrasound, planar gamma camera imaging, single-photon emission computed tomography (SPECT), positron emission tomography (PET), other nuclear medicine-based imaging, optical imaging using visible light, optical imaging using luciferase, optical imaging using a fluorophore, other optical imaging, imaging using near infrared light, or imaging using infrared light.

In some examples, an imaging agent is tested using an in vitro or in vivo assay prior to use in humans, e.g., using a model described herein.

### Samples

To the extent that the method of the present disclosure is performed in vitro, on an isolated tissue sample, rather than as an in vivo based screen, reference to “sample” should be understood as a reference to any sample of biological material derived from an animal such as, but not limited to, a body fluid (e.g., blood or synovial fluid or cerebrospinal fluid), cellular material (e.g. tissue aspirate), tissue biopsy specimens or surgical specimens.

The sample which is used according to the method of the present disclosure may be used directly or may require some form of treatment prior to use. For example, a biopsy or surgical sample may require homogenization or other form of cellular dispersion prior to use. Furthermore, to the extent that the biological sample is not in liquid form, (if such form is required or desirable) it may require the addition of a reagent, such as a buffer, to mobilize the sample.

As will be apparent from the preceding description, such an assay may require the use of a suitable control, e.g. a normal or healthy individual or a typical population, e.g., for quantification.

As used herein, the term “normal individual” shall be taken to mean that the subject is selected on the basis that they do not have abnormal numbers of RANK-L expressing cells or abnormal levels of Tweak.

A “healthy subject” is one that has not been diagnosed as suffering from a condition, e.g., an RANK-L-mediated condition and/or is not at risk of developing the condition.

Alternatively, or in addition, a suitable control sample is a control data set comprising measurements of the marker being assayed for a typical population of subjects known not to suffer from a condition.

In one example, a reference sample is not included in an assay. Instead, a suitable reference sample is derived from an established data set previously generated from a typical population. Data derived from processing, analyzing and/or assaying a test sample is then compared to data obtained for the sample population.

### Pharmaceutical Compositions

Binding molecules of the disclosure (syn. active ingredients) are useful for formulations into a pharmaceutical composition for parenteral, topical, oral, or local administration, aerosol administration, or transdermal administration, for prophylactic or for therapeutic treatment. The pharmaceutical compositions can be administered in a variety of unit dosage forms depending upon the method of administration. For example, unit dosage forms suitable for oral administration include powder, tablets, pills, capsules and lozenges.

The pharmaceutical compositions of this disclosure are useful for parenteral administration, such as intravenous administration or subcutaneous administration or administration into a body cavity or lumen of an organ or joint. The compositions for administration will commonly comprise a solution of the binding protein of the disclosure dissolved in a pharmaceutically acceptable carrier, such as an aqueous carrier. A variety of aqueous carriers can be used, e.g., buffered saline and the like. The compositions may contain pharmaceutically acceptable carriers as required to approximate physiological conditions such as pH adjusting and buffering agents, toxicity adjusting agents and the like, for example, sodium acetate, sodium chloride, potassium chloride, calcium chloride, sodium lactate and the like. The concentration of binding molecules of the present disclosure in these formulations can vary widely, and will be selected primarily based on fluid volumes, viscosities, body weight and the like in accordance with the particular mode of administration selected and the patient's needs. Exemplary carriers include water, saline, Ringer's solution, dextrose solution, and 5% human serum albumin. Nonaqueous vehicles such as mixed oils and ethyl oleate may also be used. Liposomes may also be used as carriers. The vehicles may contain minor amounts of additives that enhance isotonicity and chemical stability, e.g., buffers and preservatives.

A binding molecule of the disclosure can be formulated for parenteral administration, e.g., formulated for injection via the intravenous, intramuscular, sub-cutaneous, transdermal, or other such routes, including peristaltic administration and direct instillation into a tumor or disease site (intracavity administration). The preparation of an aqueous composition that contains the compounds of the present disclosure as an active ingredient will be known to those of skill in the art.

Suitable pharmaceutical compositions in accordance with the disclosure will generally include an amount of the binding protein of the present disclosure admixed with an acceptable pharmaceutical carrier, such as a sterile aqueous solution, to give a range of final concentrations, depending on the intended use. The techniques of preparation are generally known in the art as exemplified by Remington's Pharmaceutical Sciences, 16th Ed. Mack Publishing Company, 1980.

Upon formulation, compounds of the present disclosure will be administered in a manner compatible with the dosage formulation and in such amount as is therapeutically/prophylactically effective. Suitable dosages of compounds of the present disclosure will vary depending on the specific compound, the condition to be treated and/or the subject being treated. It is within the ability of a skilled physician to determine a suitable dosage, e.g., by commencing with a sub-optimal dosage and incrementally modifying the dosage to determine an optimal or useful dosage.

Exemplary dosages and timings of administration will be apparent to the skilled artisan based on the disclosure herein.

A pharmaceutical composition of the present disclosure may comprise an additional active agent selected from the group consisting of bisphosphonates, active vitamin D3, calcitonin and derivatives thereof, hormone preparations such as estradiol, SERMs (selective estrogen receptor modulators), ipriflavone, vitamin K2 (menatetrenone), calcium preparations, PTH (parathyroid hormone) preparations, nonsteroidal anti-inflammatory agents, soluble TNF receptor preparations, anti-TNF-[alpha] binding molecules, antibodies or functional fragments of the antibodies, anti-PTHrP (parathyroid hormone-related protein) binding molecules, antibodies or functional fragments of the antibodies, IL-1 receptor antagonists, anti-IL-6 receptor binding molecules, antibodies or functional fragments of the antibodies, anti-RANK-L binding molecules, antibodies or functional fragments of the antibodies and OCIF (osteoclastogenesis inhibitory factor).

### Methods of Treatment

Methods are provided herein for treating a bone disorder comprising administering a therapeutically effective amount of a binding molecule of the present disclosure.

In one example, methods are provided for treating a bone disorder comprising administering a therapeutically effective amount of a binding molecule and another therapeutic agent. The additional therapeutic agent may be administered in a therapeutically effective amount.

The bone disorder may be a disorder characterized by a net bone loss, including but not limited to, osteopenia and osteolysis. In certain examples, treatment with a binding molecule is used to suppress the rate of bone resorption. Therefore, treatment may be used to reduce the rate of bone resorption where the resorption rate is above normal, or to reduce bone resorption to below normal levels in order to compensate for below normal levels of bone formation.

Conditions which may be treated with binding molecules of the present disclosure include, but are not limited to, the following: Osteoporosis, including, but not limited to, primary osteoporosis, endocrine osteoporosis (including, but not limited to, hyperthyroidism, hyperparathyroidism, Cushing's syndrome, and acromegaly), hereditary and congenital forms of osteoporosis (including, but not limited to, osteogenesis imperfecta, homocystinuria, Menkes' syndrome, Riley-Day syndrome), and osteoporosis due to immobilization of extremities; Paget's disease of bone (osteitis deformans) in adults and juveniles; Osteomyelitis, i.e., an infectious lesion in bone, leading to bone loss; Hypercalcemia, including, but not limited to, hypercalcemia resulting from solid tumors (including, but not limited to, breast, lung and kidney) and hematologic malignacies (including, but not limited to, multiple myeloma, lymphoma and leukemia), idiopathic hypercalcemia, and hypercalcemia associated with hyperthyroidism and renal function disorders; Osteopenia, including but not limited to, osteopenia following surgery, osteopenia induced by steroid administration, osteopenia associated with disorders of the small and large intestine, and osteopenia associated with chronic hepatic and renal diseases; Osteonecrosis, i.e., bone cell death, including, but not limited to, osteonecrosis associated with traumatic injury, osteonecrosis associated with Gaucher's disease, osteonecrosis associated with sickle cell anemia, osteonecrosis associated with systemic lupus erythematosus, osteonecrosis associated with rheumatoid arthritis, osteonecrosis associated with periodontal disease, osteonecrosis associated with osteolytic metastasis, and osteonecrosis associated with other condition; and Loss of cartilage and joint erosion associated with rheumatoid arthritis.

In certain embodiments, an amino acid or polypeptide of the disclosure may be used alone or with at least one additional therapeutic agents for the treatment of bone disorders. Exemplary additional therapeutic agents that may be administered with a binding molecule include, but are not limited to, the bone morphogenic factors designated BMP-1 through BMP-12; transforming growth factor-[3 (TGF-[3) and TGF-[3 family members; interleukin-1 (IL-1) inhibitors, including, but not limited to, IL-ira and derivatives thereof and Kineret™; anakinra, TNFa inhibitors, including, but not limited to, soluble TNFa receptors, Enbrel™ etanercept, anti-TNFa antibodies, Remicade™, infliximab, and D2E7 antibodies; parathyroid hormone and analogs thereof; parathyroid related protein and analogs thereof; E series prostaglandins; bisphosphonates (such as alendronate and others); bone-enhancing minerals such as fluoride and calcium; non-steroidal anti-inflammatory drugs (NSAIDs), including, but not limited to, COX-2 inhibitors, such as Celebrex™, celecoxib, and Vioxx™; refecoxib, immunosuppressants, such as methotrexate or leflunomide; serine protease inhibitors, including, but not limited to, secretory leukocyte protease inhibitor (SLPI); IL-6 inhibitors (including, but not limited to, antibodies to IL-6), IL-8 inhibitors (including, but not limited to, antibodies to IL-8); IL-18 inhibitors (including, but not limited to, IL-18 binding protein and IL-18 antibodies); interleukin-1 converting enzyme (ICE) modulators; fibroblast growth factors FGF-1 to FGF-10 and FGF modulators; PAF antagonists; keratinocyte growth factor (KGF), KGF-related molecules, and KGF modulators; matrix metalloproteinase (MMP) modulators; Nitric oxide synthase (NOS) modulators, including, but not limited to, modulators of inducible NOS; modulators of glucocorticoid receptor; modulators of glutamate receptor; modulators of lipopolysaccharide (LPS) levels; and noradrenaline and modulators and mimetics thereof.

In certain embodiments, an amino acid or polypeptide of the invention is used with particular therapeutic agents to treat various inflammatory conditions, autoimmune conditions, or other conditions with attendant bone loss. For example, in view of the condition and the desired level of treatment, two, three, or more agents may be administered.

Such agents may be provided together by inclusion in the same formulation. For example, such agents and a binding molecule may be provided together by inclusion in the same formulation.

In certain embodiments, an amino acid or polypeptide of the invention is used with particular therapeutic agents to treat various cancers, specifically bone metastasis.

In some embodiments, such agents may be provided together by inclusion in a treatment kit. Alternatively, such agents may be provided separately. In certain embodiments, when administered by gene therapy, the genes encoding protein agents and/or an amino acid or polypeptide or binding molecule may be included in the same vector. The genes encoding protein agents and/or an amino acid or polypeptide or binding molecule may be under the control of the same promoter region, or the genes encoding protein agents and/or an amino acid, polypeptide or binding molecule may be in separate vectors.

### EXAMPLES

The present disclosure includes the following non-limiting examples.

### Methods

### Materials and Reagents

RAW264.7 cells are from the American Type Culture Collection. Minimum essential medium (a-MEM), L-glutamine and penicillin-streptomycin (P/S) are from the media laboratory at Harry Perkins Institute of Medical Research (Australia). Fetal bovine serum (FBS, 16000044) and Human RANKL recombinant protein (PHP0034) are from Gibco. Goat anti-mouse (ab6789) immunoglobulin G (IgG) H&L (horseradish peroxidase [HRP]) and Goat anti-rabbit (ab6721) immunoglobulin G (IgG) H&L (HRP) were purchased from Abeam. Antibodies to NFATc1 (sc-7294), v-ATPase subunit d 2 (Atp6v0d2) (sc-517031), cathepsin k (sc-48353) and [3-actin (sc-47778) were obtained from Santa Cruz. Antibody c-Fos (CST2250s), HO-1 (D60G11) and Catalase (D5N7V) were purchased from Cell Signalling Technology. Glutaraldehyde solution (25%) was purchased from Fisher Scientific. CellTiter 96 Aqueous One Solution Cell Proliferation Assay (MTS) Kit (G3580), Luciferase assay system (E1501) and the reverse transcription system were brought from Promega. Rhodamine-phalloidin, Fluo-4, AM (F14201), Purelink RNA mini kit (12183018A), Trizol (15596026), PowerUp SYBR Green Master Mix (A25918), centrifuge tubes and flasks were brought from Thermo Fisher. Western Lightning Ultra (NEL112001EA) was purchased from PerkinElmer. SYPRO® Orange Protein Gel Stain (S5692), cell culture plates, collagen-coated plates, osteo assay surface multiple well plates (CLS3987-4EA), primers, transwell polycarbonate membrane cell culture 8.0 μm inserts (CLS3422) and all other chemicals were purchased from Merck.

### I-Body Engineering

The i-body library, generated by AdAlta Limited, was used to generate binders against human RANKL recombinant protein. This library, derived from human neural cell adhesion molecule 1 (NCAM), incorporates two patented binding regions (International Patent AU2005/000789; WO/2005/118629) and was cloned in-frame with the gene Ill of the bacteriophage M13KO7 into the pHENH6 vector. This phagemid was transformed into TG-1 E. coli was used to pan against RANKL according to methods of Griffiths et. al. (2016), J Biol Chem 291, 12641-12657. Briefly, at the beginning of each panning round the library was amplified by adding 1 ml of phage library to 10 ml of 2YT medium and incubated for 1 h at 37° C. with shaking. The culture was then inoculated into 200 ml of 2YT containing 100 μg/ml ampicillin and 1% (w/v) glucose (Sigma Aldrich). Culture was then incubated at 37° C. with shaking until the absorbance at OD600 nm was 0.4-0.6. M13KO7 helper phage particles (New England Biolabs) were added at a multiplicity of infection of 20:1 (phage to bacteria) based on the assumption that 00600 nm of 1 is equivalent to 8×10⁸ E. coli cells/ml (agilent.com biocalculators calcODBacterial). The culture was incubated for 1 h without shaking at 37° C., and then the cell pellet was collected by centrifugation at 8,000 g. The pellet was then re-suspended into 200 ml of 2YT containing 100 μg/ml ampicillin and 70 μg/ml kanamycin (Sigma Aldrich) and incubated overnight at 30° C., with shaking. The next day, supernatant was clarified twice by centrifugation at 10,000 g for 10 min and phage particles precipitated 100-fold by polyethylene glycol/NaCl precipitation according to the method of Sambrook J., E. F. Fritsch and T. Maniatis. 1989. Molecular Cloning: A Laboratory Manual, 2nd ed. CSH Laboratory Press, Cold Spring Harbor, NY and resuspended in 2 ml of PB S.

### Phage Affinity Panning

The phage displayed i-body library prepared by AdAlta as described previously (Griffiths et. al. (2016), J Biol Chem 291, 12641-12657) was used to pan on immobilised carrier free hRANKL (R&D Systems). Phage displaying the i-bodies were incubated with RANKL absorbed to NI-NTA HisSorb plates via the Histidine tag on the RANKL. The panning was carried out essentially as described in (previously (Griffiths et. al. (2016), J Biol Chem 291, 12641-12657). ELISA using phage from the final rounds of panning demonstrated that there were clones specific for RANKL. Seventeen i-bodies were expressed and characterised for binding to RANKL and not to several irrelevant proteins. ADR03 was chosen as the clone with highest specificity and affinity for RANKL.

### Phage ELISAs

To prepare loading samples, single-clone phage particles were eluted from panning rounds and precipitated into 1 ml resuspension buffer. For ELISA assay, 96-well plates were coated with lipoparticles (RANKL or null, 1 unit/well). Diluted phage particles (1:10 in 5% MPBS) were added to pre-coated ELISA plates and incubated for 60 minutes followed by five-time washes to remove unbound phage particles. Bound phages were then visualised with anti-M13-HRP antibody and substrate 3,3′,5,5′-tetramethylbenzidine.

### Surface Plasmon Resonance (SPR) Assay

Kinetic binding analysis of ADR03 with immobilized RANKL lipoparticles was performed by a BIAcore T200 instrument. Biotinylated RANKL lipoparticles and null lipoparticles, used as controls, were diluted in the running buffer (1×HBS/BSA) and immobilized onto streptavidin-containing channels. To analyse binding kinetics, serial dilutions of candidates from ADR library were injected over the bound lipoparticles, where the association and dissociation phases were monitored for 60 and 600 seconds respectively. A control measurement was also included for referencing. All bound i-bodies were dissociated within 600 seconds, therefore, no regeneration of RANKL lipoparticle surface was needed between injection cycles.

### Homology Modelling, Protein-Protein Docking and the Calculation of Theoretical Pl

ADR03 structure modelling was done using the Modweb server (Version: r265) in the default setting using the ADR3 sequence and the crystal structure of 21HS (PDB: 5AEA). The predicted ADR03 structure was then docked to human RANKL (PDB: 5BNQ) using the protein-protein docking program ClusPro 2.0, mimicking the interaction between ADR3 and human RANKL (Desta, I. T., et al., (2020) Performance and Its Limits in Rigid Body Protein-Protein Docking. Structure 28, 1071-1081 e1073). Balanced coefficient weights were generated for the top 10 docking models, showing the cluster scores for evaluation. Coefficient weights: E=0.40Erep+−0.40Ea11+600Eelec+1.00EDARs. Acquired in silica structures were visualised via the Pymol Molecular Graphics System (Version: 2.2.0, Schrodinger, LLC). The ProtParam tool (Expasy) was used to calculate the theoretical pl of the designated molecules (Wilkins, M. R., et al., (1999) Protein identification and analysis tools in the ExPASy server. Methods Mol Biol 112, 531-552).

### Thermal Shift Assay (Differential Scanning Fluorimetry, DSF)

Thermal shift assays were carried out in a LightCycler® 480 real-time PCR system (Roche Life Science, Germany) using a 384 well PCR plate. The excitation and emission filters were set at 483 nm and 640 nm respectively. The protocol for the assay was adapted from Niesen et al. (2007) The use of differential scanning fluorimetry to detect ligand interactions that promote protein stability. Nat Protoc 2, 2212-2221). The protein was equilibrated in PBS buffer which had been adjusted to pH 4.0-9.0 in 1.0 intervals using HCl or NaOH. The assay used 20 μM protein and 5× Sypro Orange per well with a total volume of 10 μL. The PCR plate was sealed with an optical seal and centrifuged at 1000×g for 2 minutes after the protein and dye were added. The assays were carried out in quadruplicate. A temperature gradient of 20° C. to 95° C. with ramp rate of 0.03° C./s and 20 acquisitions per ° C. was used for the assay. The data was analysed using the “Tm calling” feature of the LightCycler® 480 software which plots the temperature against the first derivative of fluorescence intensity over temperature.

### Animal Cell Isolation

Bone marrow macrophages (BMMs) were flushed out from the femur and tibia of 12-week C57BL/6J mice using the method under the approval of the University of Western Australia Animal Ethics Committee (RA/3/100/1601) and then maintained in a-MEM containing 10% FBS, 1% P/S and M-CSF (complete a-MEM). To remove undesired fibroblasts, non-adherent cells were transferred to a new flask 6 hours after the dissection.

### MTS Assay

Fresh BMMs were seeded in 96-well plates at the density of 6×10³ cells/well and incubated for overnight. Varying doses of ADR03 i-body were added to BMMs for 48-hour incubation. 25 μl MTS/phenazine methosulfate mixture was then mixed with each well for 2 h. The absorbance was measured by an ELISA plate reader (BMG LABTECH GmbH).

### In Vitro Osteoclastogenesis Assay

RAW264.7 cells and fresh bone marrow cells (BMMs) were maintained in a-MEM (10% FBS and P/S) and complete a-MEM respectively under the culture condition of 37° C. with 5% CO2. For functional assay, RAW264.7 cells and BMMs were seeded to 96 well plate at the density of 6×10³ cells/well and induced by 40 ng/ml hRANKL. Media was replaced every two days until osteoclasts formed in the positive control group. Cells were then fixed in 2.5% glutaraldehyde at room temperature (RT) for 10 min and stained for tartrate resistant acid phosphatase (TRAcP). TRAcP-positive cells were imaged by a Nikon microscope (Nikon Corporation) and quantified via imageJ (NIH).

### Podosome Belt Staining

BMMs were seeded in a 24-well plate with 13-mm coverslips and induced by 40 ng/ml hRANKL with or without i-bodies, until the formation of mature osteoclasts. Cells were fixed in 4% paraformaldehyde at RT for 10 min and permeabilized with 0.1% Triton X-100 for 5 min before 1-hour incubation with 3% bovine serum albumin (BSA) PBS for blocking. After washing with 0.2% BSA-PBS, cells were incubated with rhodamine-phalloidin (1:300 in 0.2% BSA-PBS) for 2 h at RT. Cells were then gently washed in 0.2% BSA-PBS and PBS four times before staining with 4′,6-diamidino-2-phenylindole (1:10,000 in PBS) for 5 min. After washing with PBS, stained cells were visualized via a NIKON A1Si Confocal Microscope (Nikon Corporation).

### Luciferase Reporter Assay

To study the activity of NFATc1, 5×10⁴ RAW264.7 cells stably transfected with NFATc1 luciferase reporter gene described in (Qiu, H., et al., Cell Physiol 236, 2800-281651) were seeded into 48-well plates for overnight. Cells were then induced by 40 ng/ml hRANKL treated with or without i-bodies for 24 hours. Luciferase activity was measured using the Promega Luciferase Assay System according to the manufacturer's instructions.

### In Vitro Hydroxyapatite Resorption Assay

BMMs were cultured on 6-well collagen-coated plates in the presence of 40 ng/ml hRANKL for 5 days. Upon the formation of small osteoclasts, cells were trypsinized and transferred to 96-well osteo assay plates for a 2-day culture in the presence of 40 ng/ml hRANKL with or without i-bodies. At day 8, half the wells of each group were fixed in 2.5% glutaraldehyde for TRAcP staining, while the other half were bleached for visualisation of resorptive pits. Images were captured by a Nikon microscope (Nikon Corporation) and quantified through ImageJ (NIH).

RNA Extraction and cDNA Synthesis

BMMs were cultured in 6-well plates with complete a-MEM at a density of 1×10⁵ cells/well. Cells were treated with 40 ng/ml hRANKL with or without i-bodies for 5 days until osteoclasts formed in positive control. Cells were then lysed for total RNA extraction using Trizol and Purelink RNA mini kit in accordance with the manufacturer's protocol. For cDNA synthesis, reverse transcription PCR was performed using Promega reverse transcription system.

### Real-Time Polymerase Chain Reaction (PCR)

SYBR Green PCR Master Mix was used for real-time PCR (rtPCR). The cycling parameters for PCR were set as follows: 94° C. for 5 min, followed by 30 cycles of 94° C. (40 s), 60° C., (40 s); 72° C. (40 s) and a 5-min elongation step at 72° C. rtPCR was performed using primers as described: calcitonin gene-related peptide type 1 receptor (Calcr1; Forward: 5′-TGGTTGAGGTTGTGCCCA-3′, SEQ ID NO: 17; Reverse: 5′-CTCGTGGGTTTGCCTCATC-3′, SEQ ID NO: 18), c-Fos (Forward: 5′-GCGAGCAACTGAGAAGAC-3′, SED ID NO:19; Reverse: 5′-TTGAA ACCCGAGAACATC-3′, SEQ ID NO: 20), nuclear factor of activated T-cells, cytoplasmic 1 (Nfatc1; Forward: 5′-CA ACGCCCTGACCACCGATAG-3′, SEQ ID NO: 21; Reverse: 5′-GGCTGCCTTCCGTCTCATAGT-3′, SEQ ID NO: 22), ATPase, H+ transporting, lysosomal VO subunit D2 (Atp6vOd2; Forward: 5′-GTGAGACCTTGGAA GACCTGAA-3′, SEQ ID NO: 23; Reverse: 5′-GAGAAATGTGCTCAGGGGCT-3′, SEQ ID NO: 24), tartrate-resistant acid phosphatase (Acp5; Forward: 5′-TGTGGCCATCTTTATGCT-3′, SEQ ID NO: 25; Reverse: 5′-GTCATTTCTTTGGGGCTT-3′, SEQ ID NO: 26), cathepsin K (Ctsk; Forward: 5′-GGGAGAAAAACCTGAAGC-3′, SEQ ID NO: 27; Reverse: 5′-ATTCTGGGGACTCAGAGC-3′, SEQ ID NO: 28), matrix metallopeptidase 9 (Mmp9; Forward: 5′-CGTGTCTGGAGATTCGACTTGA-3′, SEQ ID NO: 29; Reverse: 5′-TTGGAAACTCACACGCCAGA-3′, SEQ ID NO: 30) and -actin (ACTB; Forward: 5′-AAGATCAAGATCATTGCTCCTCCT-3′, SEQ ID NO: 31, Reverse: 5′-AGCTCAGTAACAGTCCGCCT-3′, SEQ ID NO: 32). rtPCR reaction results were read on a ViiA 7 Real-time PCR machine (Applied Biosystems). The Ct values of target genes were normalized to the Ct value of ACTB to give a ΔCt value, in which the data of the experimental groups was further normalized to the control groups to obtain ΔΔCt. Three independent cultures were carried out and all experiments were performed in triplicate.

### Western Blotting

Fresh BMMs were induced by 40 ng/ml hRANKL with or without i-bodies in 6 well plates (1×10⁵ cells/well) for 5 days until the formation of mature osteoclasts in the positive control. Cells were lysed in RIPA buffer and boiled for 5 mins with 4× loading buffer. Samples were resolved on 10% sodium dodecyl sulfate (SDS) denatured acrylamide gels and electro blotted onto 0.2 um nitrocellulose membranes. Membranes were then blocked with 5% (wt/vol) nonfat milk powder in TBST (10 mM Tris, pH 7.5, 150 mM NaCl, 0.1% [vol/vol] Tween-20) and incubated with primary antibodies diluted (1:500-1000) in TBST containing 1% BSA (wt/vol). HRP-conjugated secondary antibodies were diluted (1:3300) in 1% BSA (wt/vol) in TBST. Proteins were visualised by western lighting from a PerkinsElmer and an ImageQuant LAS4000 (GE).

### Calcium Flux Assay

In general, starved fresh BMMs were seeded in 48-well plates (1×10⁴ cells/well) for overnight before being treated with 40 ng/ml hRANKL with or without i-bodies for 24 hours. Cells were gently washed with assay buffer (HANKS buffer containing 1 mM probenecid and 1% FBS) and labelled by calcium indicator Fluo-4 AM (in assay buffer supplemented with 20% dimethyl sulfoxide-diluted pluronic-F127 (wt/vol)) according to the manufacturer's guidance (Molecular probes; Thermo Fisher Scientific). Intracellular Ca²+ was subsequently visualized through inverted fluorescent microscopy (Nikon) at 488 nm wavelength. Images were taken every 2 s lasting for 3 min for each well. Further analysis was conducted using the Nikon Basic Research Software.

### Transwell Assay

HUVECs were starved for 24 hours before seeded to the upper chambers of the polycarbonate membrane cell culture 8.0 μm inserts at the density of 10⁵ cells/ml. The bottom chambers were filled up with DMEM (1.0% FBS) containing PBS, i-body Ctrl, 0.5 μM ADR03 or 1.0 μM ADR03. HUVECs were allowed to cross the member for 24 hours before fixed in 2.5% glutaraldehyde solution. Fixed cells were then stained by 0.5% crystal violet. Cells in the upper chambers were removed, while cells remaining in the lower chambers were visualised under microscopy and quantified through ImageJ.

### Statistical Analysis and Graph Preparation

The statistics was carried out by paired Students' t test with significance taken at least p<0.05. GraphPad Prism 6 was used to present data. At least three independent experiments were conducted.

### Example 1 Identification of RANK-L Blocking i-Bodies

The principles learnt from shark IgNAR antibody structures can be successfully applied to the generation of binding repertoires of human I-set immunoglobulins which is further described in WO2005118629. Shark IgNAR antibodies are structurally close to I-set domain immunoglobulins such as Domain 1 of NCAM. The modified Domain 1 of NCAM is referred to as the i-body scaffold. Using this scaffold a library of amino acids and polypeptides is created and displayed on phage for screening against particular targets for specific binders to that target. Such libraries are anticipated to primarily contain variability in the CDR1 and CDR3 analogous regions.

An i-body library was created which had a random amino acid sequence in the CDR1 region (represented by XXXXXX in FIG. 1A) and in the CDR3 region (represented by Y′n), wherein n (the number of amino acids in the random CDR3 sequence) is varied randomly between 10 and 20 amino acids in length and sequence.

I-bodies displayed on phage were selected against human RANK-L (hRANK-L) following incubation of the i-body library and hRANK-L captured on plates or beads. Extensive washing was completed to remove non-specific binders. Enrichment to the hRANKL was observed and single colonies were picked and grown. The sequence of the i-body scaffold remains the same except for the specific sequences of the CDR1 and CDR3 regions.

### Example 2 Identification and Characterisation of ADR03

### 2.1. I-Body Expression and Purification

I-bodies were expressed and purified using an E. coli expression system. I-bodies were expressed with various affinity tags including FLAG or penta-HIS. I-bodies were purified from the periplasmic fraction and the cytoplasm using the various tags using anti-FLAG resin or Ni-NTA. The inventors produced 17 i-body clones which bound RANKL. Based on phage ELISA and SPR results, ADR03 was identified as the best candidate which exhibited the highest binding affinity to human RANKL (Ko=13 nM).

The ADR03 i-body sequence is shown in FIGS. 1A and 1B. The sequences of the clones are summarised in FIG. 1B. The sequence identity of the clones to ADR03 is summarised in Table 1 below:

TABLE 1
RANKL i--body clones

| Clone | Sequence identity (%) to ADR03 |
|---|---|
| ADR1 | 80 |
| ADR2 | 84 |
| ADR4 | 86 |
| ADR5 | 83 |
| ADR6 | 87.5 |
| ADR7 | 83 |
| ADR8 | 87 |
| ADR9 | 84 |
| ADR10 | 82.5 |
| ADR11 | 84 |
| ADR12 | 85 |
| ADR13 | 86 |
| ADR14 | 87 |
| ADR15 | 83 |
| ADR16 | 88 |
| ADR17 | 86 |

TABLE 2
ADR03 binding loop sequence

| | | | Length of | |
|---|---|---|---|---|
| | CDR1 | CDR3 | CDR3 | |
| Name | Sequence | Sequence | (amino acids) | SEQ ID NO. |
| ADR03 | AHTVES | VASARRGFGWVYPH | 14 | 11,12,13 |

ADR03 in the “lm7-FH” format was tagged with the ˜11.9 kDa lm7-FLAG-His6 fusion protein and then cloned, expressed and purified from E. coli as described in Table 3 below.

TABLE 3
Expression and purification of ADR03

| Construct | PGC ADR03 iM7-FH |
|---|---|
| Sequence | LQVDIVPSQGEISVGESKFFLCQVAGAHTVESISWFSPNGE |
| | KLTPNQQRIGVVWNDDSSSTLTIYNANIDDAGIYKCVVVA |
| | SARRGFGWVYPHEATVNVKIFQAAAGSGGGGSGGRASE |
| | LKNSISDYTEAEFVQLLKEIEKENVAATDDVLDVLLEH |
| | FVKITEHPDGTDLIYYPSDNRDDSPEGIVKEIKEWRAANG |
| | KPGF KQGAAADYKDDDDKAAAHHHHHH |
| Number of amino acids | 224 |
| Expected size | 21 kDa (including the lm7 His tag) |
| Theoretical pl | 4.96 |
| Expression host | TG1 |
| Stating material | E. coli pellet from 2 x 1L expression cultures |
| Procedures | Spheroplast preparation Comparison of IMAC Roche |
| | Complete and GE HisTRAP resins |

The construct was produced in four small scale fed-batch fermentation processes (see Table 4 below). Peaks 1 and 2 were combined to determine the total i-body yields. The total yield from each process was calculated assuming a final volume (at harvest) of 2 Litres. This is typically the final volume after addition of the seed culture, feed and base for pH control.

TABLE 4
Production of ADR03

| | Yield from | Volume | i-body | i-body |
|---|---|---|---|---|
| | periplasmic | of ferment | yield | yield |
| | extraction | processed | (mg/L of | (ng/ |
| Batch Number | (mg) | (mL) | culture) | ferment) |
| 051212-Adalta- | 40.8 | 625 | 65.3 | 131 |
| 02-T-01 | | | | |
| 051212-Adalta- | 22.2 | 625 | 35.5 | 71 |
| 02-T-02 | | | | |
| 121212-Adalta- | 25.7 | 512 | 50.1 | 100 |
| 02-T-03 | | | | |
| 121212-Adalta- | 36.6 | 510 | 71.7 | 143 |
| 02-T-04 | | | | |

ADR03-lm7-FH was analysed by size exclusion chromatography under the following conditions:

### Analytical SEC-HPLC:

Instrument: Agilent 1100 HPLC running ChemStation Software
Injection volume: 20p 1
Wavelength collected: 214 nm
Column: Agilent AdvanceBio 4.6 mm×300 mm (Batch #0006330244)
Running Buffer: 150 mM phosphate, pH7.0
Run time: 15 mins
Standards: Agilent AdvanceBio

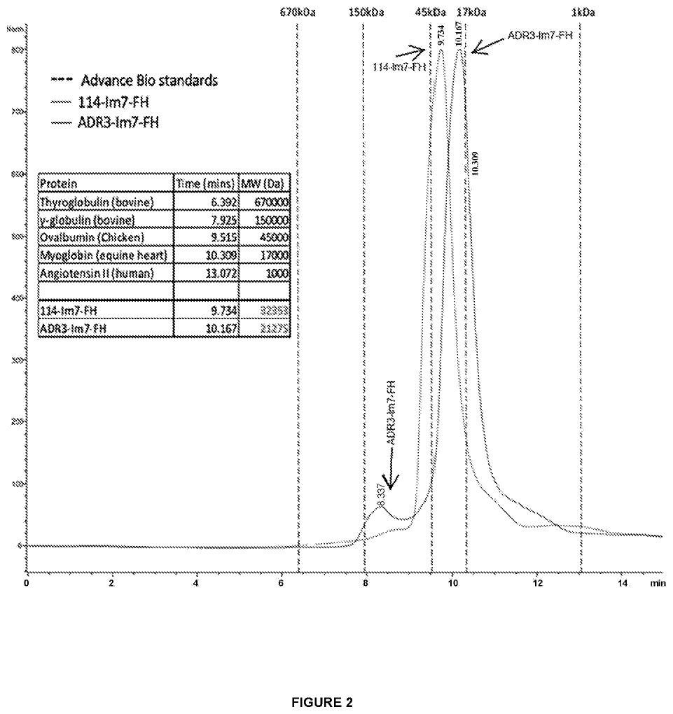

Results of this purification are shown in FIG. 2.

### 2.2 ADR03 Affinity to Human RANK-L

Measurement of the kinetics of ADR03 binding to human RANK-L was carried out by immobilising human RANKL onto a research grade CMS sensor chip using standard amine coupling. Each of three surfaces is first activated for seven minutes using a 1:1 mixture of 0.1 mM N-hydroxysuccinimide (NHS) and 0.4 mM 1-ethyl-3-(3-dimethylaminopropyl)-carbodimide (EDC). Then, the RANK-L sample is diluted 1- to 50-fold in 10 mM sodium acetate, pH 4.0, and exposed to the activated chip surface for different lengths of time (ten seconds to two minutes) to create three different density surfaces of RANK-L. Each surface is then blocked with a seven-minute injection of 1 M ethanolamine, pH 8.2. RANK-L is diluted 100-fold and injected for different amounts of time to be captured at three different surface densities (60 RU, 45 RU, 12 RU; Response Unit (RU) is termed by Biacore and relates to target molecule per surface area) onto a streptavidin-containing sensor chip. The experiments can be performed on a Biacore® 2000 or T100 optical biosensor. ADR03 is supplied at approximately 100 μg/mL and tested in a 3-fold dilution series in Sample Running Buffer over the three RANK-L surfaces. Each of ADR03 concentrations (five, 3-fold dilutions of RANK-L-coupled sensors) is tested three times to assess reproducibility of the assay. Each test is injected at a flow rate of 100 μL/minute for 60 seconds, followed by a three-minute dissociation phase. Bound ADR03 can then be removed using a five-second pulse with sensor regeneration solution. All data is usually collected at a temperature-controlled 20° c. The kinetic responses for the ADR03 injections can be analyzed using the non-linear least squares analysis program CLAMP (Myszka, D. G. and Morton, T. A. (1998) Trends Biochem. Sci., 23: 149-150). Calculations of multivalent interactions can be determined using a model to fit the avidity of the bivalent interaction of ADR03 with RANK-L (Drake et al. (2004) Anal. Biochem., 328: 35-43; and Muller et al., (1998) Anal. Biochem. 261: 149-158).

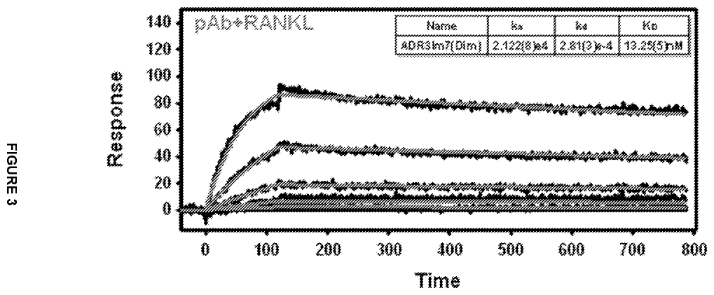

ADR03 was shown in this assay to bind specifically to human RANK-L immobilized on a SPR chip with an affinity or Ko of 13.2.:t. 3.9 nM, a Ka of 2.9.:t. 0.1×10⁴ and a Kd of 2.9.:t. 0.1×10⁴ (FIG. 3).

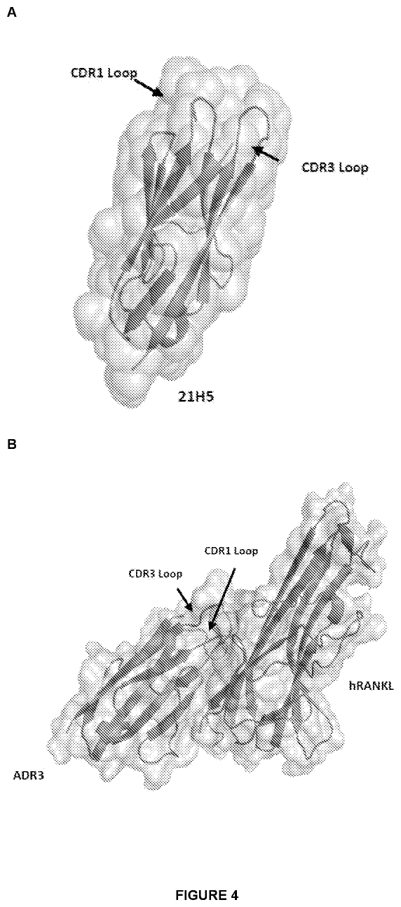

The crystal structure of 21HS, lg domain of NCAM (PDB: 5AEA), is shown in FIG. 4a, along with two colour-indicated regions (CDR1 and CDR3) where the long binding loops were inserted, enabling tight binding to disease-causing targets. The inventors further provided a best-fit computational model for the interaction between predicted ADR03 (FIG. 4b and hRANKL (PDB: 5BNQ) on the basis of thermodynamics and homology modelling, in which the model clearly showed that CDR1 and CDR3 in ADR3 were intensively involved in the interaction (FIG. 4b).

2.3 the Thermal Stability of i-Body ADR3

It is known that a healthy body temperature ranges from 97° F. (36.1° C.) to 99° F. (37.2° C.) with a pH value between 7.35 and 7.45. To work effectively in humans, therapeutic goods must be stable while traveling through the body. Hence, the inventors conducted Thermal Shift Assays (Differential Scanning Fluorimetry, DSF) on ADR03, to test its stability in a wide range of temperatures and pHs. Sypro Orange dye was used in this assay to monitor the thermal denaturation of ADR03. As proteins unfold, their hydrophobic core residues are exposed to the solution where the dye binds to those residues and emits a fluorescent signal (Lo, M. C., et al., (2004) Evaluation of fluorescence-based thermal shift assays for hit identification in drug discovery. Anal Biochem 332, 153-159). The unfolding temperature (Tm) of the molecule is indicated by the maximum value of the first derivative of fluorescence intensity over temperature.

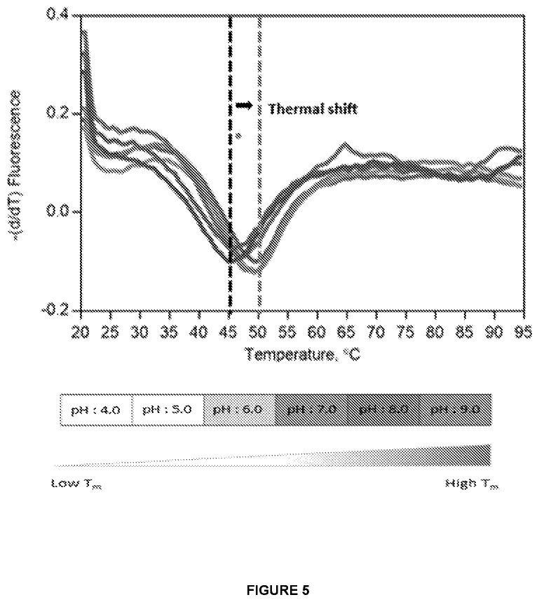

ADR03 exhibited good thermal stability across different temperatures and pHs. ADR03 was more stable from pH 7.0-9.0 ((FIG. 5). Lower stability from pH 4.0-5.0 may be due to the fact that this range is near to the theoretical pl of the molecule (4.96, calculated based on sequence using ProtParam) (Wilkins, M. R., et al; (1999) Protein identification and analysis tools in the ExPASy server. Methods Mo Biol 112, 531-552). At its pl, the net charge of the protein is zero, and resulting in aggregation and lower stability of the protein. Given the physiological temperature and pH range in normal human subjects, it is predicted that ADR03 will be sufficiently stable as a potential therapeutic agent in the human body.

### 2.4 Binding to Mouse RANK-L

Binding by ADR03 to RANK-L can be examined using Costar EIA/RIA 96-well plates coated with 75 μI/well of recombinant murine RANK-L (158-316) at 3 μg/ml in PBS. After overnight incubation at 4° C., RANK-L solutions are then removed and plates blocked with 5% chicken serum (Gibco) in PBST (PBS plus 0.05% Tween20) and incubated at room temperature for 3 hr with agitation. Plates are then washed with 1×KP wash solution (Kirkegaard-Perry Laboratories) in distilled water. ADR03 is serially diluted in PBST and added to the RANK-L-coated plates. Plates are then incubated for 7 hr at room temperature with agitation and washed and washed with 1×KP wash solution. HRP labelled Anti FLAG or anti Histidine antibodies are dilutes 1:3000 in 5% chicken serum in PBST (C-PBST) and then added to the wells. Plates are then incubated for 1 hr at room temperature with agitation and washed with 1×KP. Undiluted ABTS substrate is added and plate incubated at room temperature. Colour development is stopped after 4 min by addition of 1% SDS and measured at 405 nm.

### 2.5 Binding to TNF-a, TNF-Beta, CD40L, TRAIL

In order to confirm specificity of binding to RANK-L, ADR03 can be examined for binding to TRAIL. Costar EIA/RIA plates can be coated with anti-FLAG M2 monoclonal antibody (3 μg/ml) in PBS. After overnight incubation at 4° C., the antibody is removed and plates blocked and washed as described above. FLAG-human TRAIL (2 μg/ml in C-PBST) is added to each well and incubated for 1-2 hr, and plates washed with 1×KP wash solution. Serial dilutions of human OPG-Fc or ADR03 in C-PBST are then added to wells and plates agitated for 2 hr at room temperature. After three washes with 1×KP solution, a 1:5000 dilution of the anti-human IgG (Fc)-HRP conjugate is added to each well and the plates agitated for 1 hr. After three final washes, ABTS substrate is added and color development monitored as described above.

A similar protocol as described for TRAIL can be used to assess binding of ADR03 to TNF-a, TNF-beta and CD40L.

### Example 3 ADR03 Reduces TRAP Activity and Reduces Osteoclastogenesis in RAW264.7 Cells and Bone Marrow Macrophages (BMMs)

Osteoclastogeneis can be monitored in culture of murine RAW 264.7 macrophages which serve as osteoclast precursors, according to previously described methods (Xu J et al. (2000) J Bone Miner Res 15:2178-2186). 10⁴ cells/well in 100 μl D-MEM+5% FCS. 100p of medium containing RANK-L (60 ng/ml final concentration) and i-body (0 to 3 μg/ml final concentration) was added to each well and the cells were incubated at 37° C. for 3 days. Osteoclast precursor formation was evaluated by measuring tartrate-resistant acid phosphatase (TRAP) activity. Cells were fixed with 10% formalin for 10 minutes and 95% ethanol for 1 minute and then dried. To measure TRAP activity, 100 μl citrate buffer (50 mM pH 4.6) containing 10 mM tartrate and 1 mg/ml p-nitrophenylphosphate was added to the fixed, dried cells. After 30 minutes incubation, the enzyme reaction mixtures were transferred to another 96 well plate containing 100 μl 0.1 M NaOH in each well. Absorption was measured at 405 nm with a Molecular Devices plate reader. as previously described (Simonet W S et al. (1997) Cell 89:309-319).

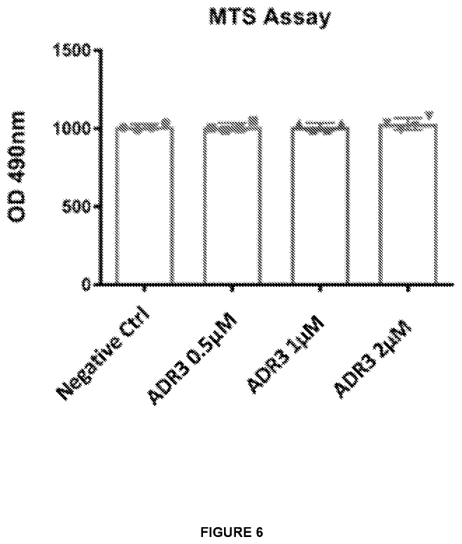

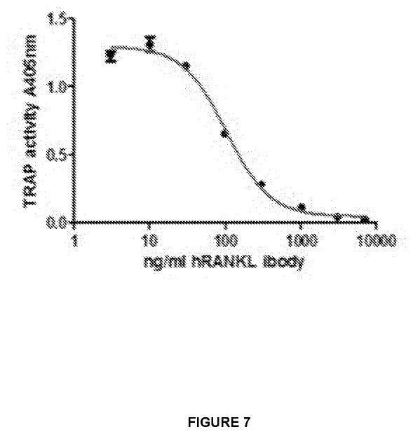

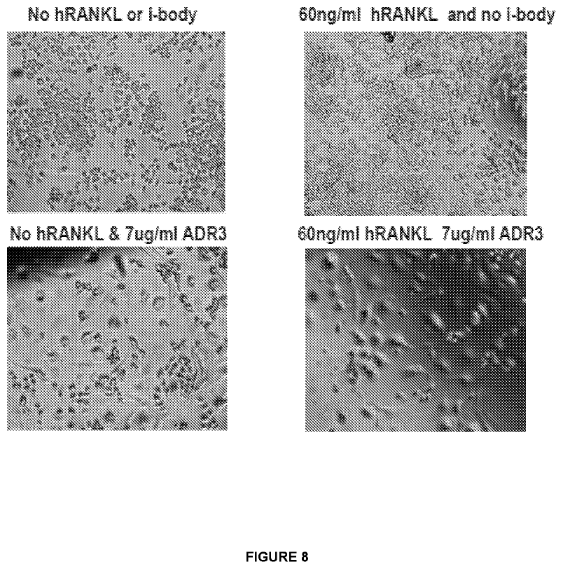

Cell proliferation assay (MTS) showed no cytotoxicity of ADR03 in BMMS within a range of concentrations (0.5-2 μM) (FIG. 6). Furthermore, ADR03 i-body blocked RANKL-induced osteoclastogenesis in RAW264.7 by inhibiting the hRANK-L-induced production of TRAP positive multinucleated cells with an ICso of 100 ng/mL or 3 nM (FIGS. 7 and 8).

In order to compare the effect of ADR03 on human and mouse RANK-L induced osteoclastogenesis, mouse RAW264.7 cells were converted into TRAP positive cells by the addition of hRANK-L (60 ng/ml) or mRANK-L (60 ng/ml). hRANK-L i-body binder ADR03 (RK i-body) inhibited both hRANK-L and mRANK-L osteoclastogenesis measured by TRAP activity, whilst the two control antibodies, hRANK-L CX (negative control i-body) and hRANK-L FF (negative control i-body) did not inhibit osteoclastogenesis (FIGS. 7 and 8). When the human and mouse results were converted to % of number of i-body controls the dose response curves were identical.

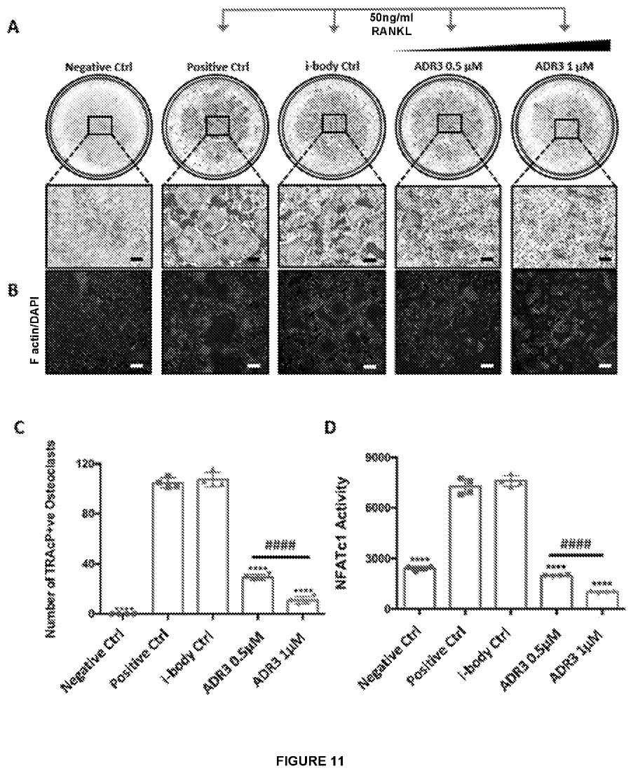

ADR3 dose-dependently suppressed osteoclast differentiation and podosome belt formation in freshly isolated BMMs (FIG. 11A-C). Nuclear factor of activated T-cells, cytoplasmic 1 (NFATc1) is a master transcriptional factor induced by RANKL for the terminal differentiation of osteoclasts, so we next examined whether the activity of NFATc1 could also be inhibited by ADR3. A robust reduction of luciferase activity in RAW264.7 cells stably transfected with NFATc1 luciferase reporter gene was observed in ADR3 treatment groups in comparison to the positive control, suggesting that ADR3 interrupted the activation of NFATc1 by RANKL in a dose-dependent manner (FIG. 11D).

### Example 4 i-bodVADR03 Suppresses Osteoc/Ast Bone Resorption

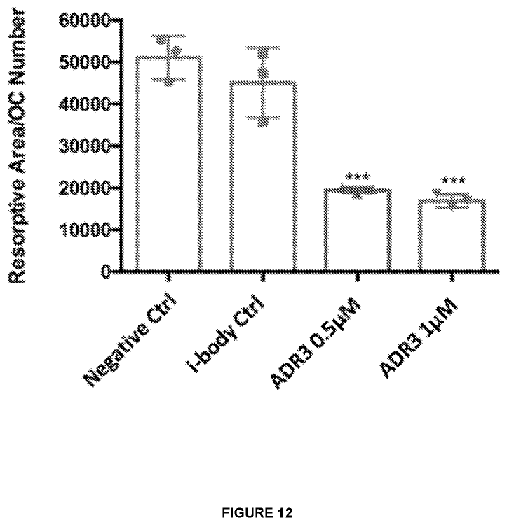

RANKL is involved not only in the formation of pre-fusion osteoclasts (pOCs), but also in the maintenance of mature osteoclast survival and bone-resorbing activity. The inventors therefore examined the in vitro effect of ADR03 on bone resorption via osteo assay plates. Compared to the no treatment group, bone resorptive area was significantly reduced after 48-hour treatment with ADR3 in two doses (0.5 μM and 1 μM), along with a subtle change in the number of TRAcP-positive cells. The ratio of total resorptive area to osteoclast number showed an evident regress of resorbing capacity for each osteoclast (FIG. 12), indicating that ADR03 hampered osteoclasts' resorptive functionality.

### Example 5 i-Body ADR3 Decreased the Expression of Osteoclast Markers Induced by RANKL Both at Gene and Protein Level

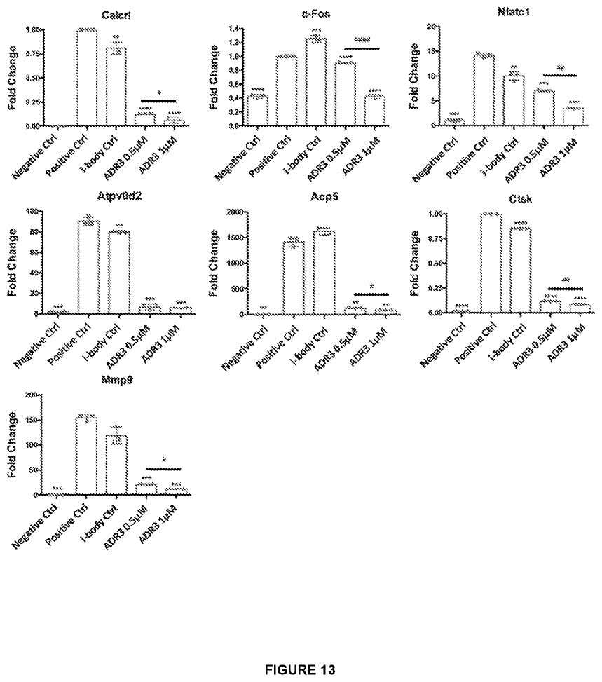

During osteoclastogenesis, the formation of RANKL-RANK complexes induces c-Fos (Proto-oncogene c-Fos) expression, initiating the induction of NFATc1 which promotes the synthesis of fusion protein, such as vacuolar (H+) ATPase Vo domain d2 isoform (Encoded by Atpv0d2 gene), and lysosomal enzymes, including tartrate-resistant acid phosphatase type 5 (Encoded by Acp5 gene), cathepsin K (Encoded by Ctsk gene) and matrix metalloproteinase-9 (Encoded by Mmp9 gene), that help to dissolve bone matrix (Matsuo, K., et al., (2004) Nuclear factor of activated T-cells (NFAT) rescues osteoclastogenesis in precursors lacking c-Fos. J Biol Chem 279, 26475-26480). qPCR showed a considerably lower expression of Calcitonin gene-related peptide type 1 receptor (Calcr1), c-Fos, Nfatc1, Atpv0d2, Acp5, Ctsk and Mmp9 in ADR3-treated groups compared to the control group (FIG. 13).

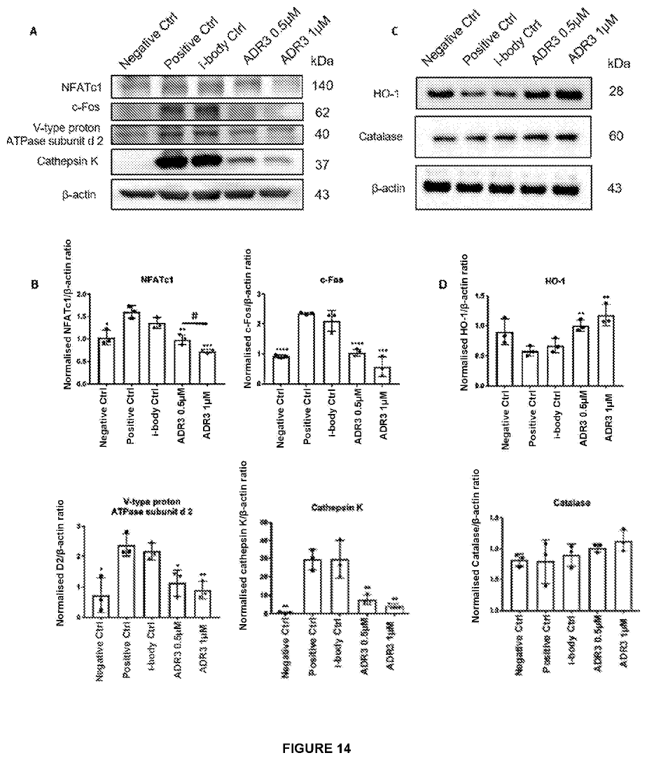

This is supported by western blotting results which showed that protein levels of NFATc1, c-Fos, Atpv0d2 and Ctsk also decreased in groups treated by ADR3 (FIG. 14A-B), consolidating the anti-catabolic effect observed in vitro. Interestingly, RANKL was reported to activate reactive oxygen species (ROSs) during osteoclast differentiation (Lee, N. K., et al., (2005) A crucial role for reactive oxygen species in RANKL-induced osteoclast differentiation. Blood 106, 852-859(24). Heme Oxygenase-1 (HO-1), an antioxidant enzyme, was suggested to be a negative regulator of osteoclastogenesis through alleviating the elevated ROSs activity (Florczyk-Soluch, U., et al., (2018) Various roles of heme oxygenase-1 in response of bone marrow macrophages to RANKL and in the early stage of osteoclastogenesis. Sci Rep 8, 10797). Similarly, another antioxidant enzyme, catalase, was also reported to inhibit ROSs production (24). Therefore, we measured the level of HO-1 and Catalase in RANKL-stimulated BMMs after ADR3 treatment and found that ADR3 robustly increased HO-1 expression (FIGS. 14C and 14D). The same trend but to a lesser effect was observed in the levels of Catalase (FIGS. 14C and 14D). Collectively, i-body ADR3 was shown to effectively suppress the expression of osteoclast makers, bone-resorptive enzymes, transcriptional factor and ROS activity both at the gene and protein levels.

### Example 6 i-Body ADR03 Treatment Does-Dependently Impaired RANKL-Induced Ca²+ Oscillations

Formation of the RANKL-RANK complex activates phospholipase Cy (PLCy) which can induce cytosolic inositol 1, 4, 5-triphosphate (IP3), releasing Ca²+ from the endoplasmic reticulum (ER) into the cytosol. RANKL-triggered Ca²+ oscillation activates CaM-dependent enzymes which facilitate the auto-amplification and translocation of NFATc1 to the nucleus (Kang, J. Y., et al., (2020) The Role of Ca(2+)-NFATc1 Signaling and Its Modulation on Osteoclastogenesis. Int J Mo Sci 21). Thus, we labelled the calcium with the fluorescence indicator Fluo-4 AM and investigated the effect of ADR03 on Ca²+ transport during RANKL-mediated osteoclastogenesis.

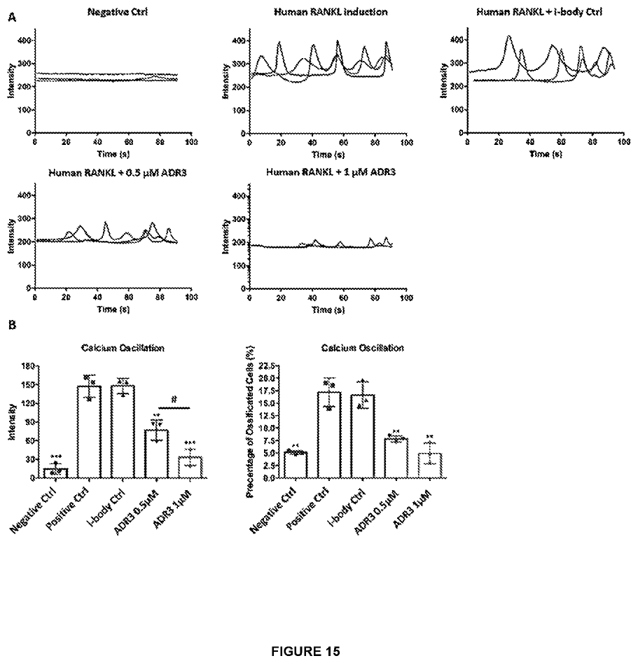

As shown in FIG. 15A, 24-hour treatment of hRANKL significantly increased Ca²+ oscillation in BMMs, while 0.5 μM and 1 μM ADR03 treatment clearly blocked the oscillation signal, flattening the fluctuation of the fluorescence signal. The quantification of maximum fluorescence change also showed a dose-dependent reduction of peak to peak values on the ADR03 treatment group, along with a decrease in the percentage of oscillations of BMMs, suggesting that both Ca²+ flux intensity of single cells and the percentage of oscillated cells were reduced (FIG. 15B).

### Example 7 i-Body ADR3 Enhanced the Migration of Human Umbilical Vein Endothelial Cells (HUVECs)

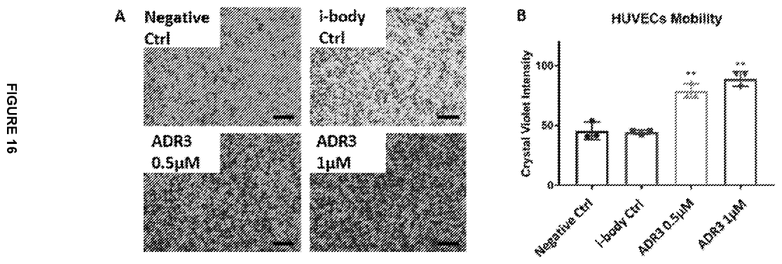

Surprisingly, ADR03 treatment significantly enhanced HUVECs migration in a dose-dependent manner (FIGS. 16A and 16B). Migration of endothelial cells is an essential part of angiogenesis, and sabotaged angiogenesis often leads to osteoporosis (Lamalice, L., et al., (2007) Endothelial cell migration during angiogenesis. Gire Res 100, 782-79429). The unexpected angiogenic effect of ADR03 may improve bone vasculature and protect bone loss in addition to suppression of osteoclast formation. Limited hRANKL expression was detected in HUVECs (Data not shown), however, whether this angiogenic effect is mediated by RANKL signalling remains elusive.

### Example 8 Effect of i-Bodies on In Vivo Mouse Models of Osteoporosis

The biological effect of ARD03 can be assessed in a mouse experimental model of osteoporosis such as an ovariectomized mouse model of postmenopausal osteoporosis. The oestrogen deficiency induced by a bilateral ovariectomy results in bone loss which present characteristics of postmenopausal osteoporosis, i.e., cancellous and endocortical bone loss. These effects result from an increase of the overall rate of bone remodeling associated with an alteration of the balance between bone formation and bone resorption, such that resorption predominates at selected skeletal sites (Jee and Yao J Musculoskelet Neuronal Interact. 1(3):193-207 (2001); Yamane et al., Bone 44(6); 1055-62 (2009)).

### Example 9 Analysis of Effect of i-Bodies on Tumor Progression In Vivo

Inhibition of tumor progression after administration of an i-body (e.g. ADR03) can be confirmed using a mouse model of breast cancer bone metastasis as described in Canon et al., Clin Exp Metastasis 25:119-129 (2008). The animal model is generated by implanting or injecting MDA-MB-231 tumor cells into mice. The methodology involves analysing MDA-MB-231 tumor cells in bone for active caspase-3 expression as a measure of apoptosis. Apoptosis can be measured in histological section of femurs/tibias by immunoassay for active caspase-3 (Cell Signalling, Danvers, MA). The total number of cells positive for caspase-3 in the entire tumor area in the bone can be recorded and the ratio of cells positive for caspase-3 staining per tumor area plotted. Osteoclasts (OC) within the tumor and on bone in contact with the tumor can be scored using TRAP stained sections (Leukocyte Acid Phosphatase kit, Sigma, St Louis, MO). TRAP staining can be scored on a scale of 0-4. It is expected that tumor cells in bones treated with i-bodies will show a significantly higher degree of apoptosis compared with control treatment and reduced TRAP staining. It is expected that the subsequent tumor growth will be significantly reduced with the addition of the anti-RANKL i-body.

### Example 10 Pharmacokinetics and Pharmacodynamics of i-Bodies

Pharmacokinetics and pharmacodynamics of i-bodies (e.g. ADR03) before and after PEGylation can be determined according to standard methods. Concentration of i-bodies can be determined in plasma or serum from a mouse as described below. Mice (4-5 mo old, approx 6/group) can be injected subcutaneously with vehicle (PBS) or with ADR03 (at doses of 0.2, 1.0 or 5.0 mg/kg). Serum or plasma is then obtained from blood which can be drawn from the retro-orbital plexus of the mice at days 1, 4 and 7 for measurement of serum TRAcP-5b (BoneTRAP® Elisa, Immunodiagnostics Systems). Serum or plasma i-body concentration can be assessed by coating 96-well polystyrene plates with 2 μg/ml hRANK-L (143-317) and overnight incubation at 4° C. or detection of the i-body through the tag. Pre-treatment serum can be used to determine non-specific binding. It is expected that single subcutaneous injections of i-body will result in dose-dependent increases in serum drug levels, which can be measured over 7 days. It is expected that the PEGylated polypeptide will have a longer half life than the non-PEGylated polypeptide.

Changes in bone resorption induced by i-bodies can be assessed by assaying serum NTx using immunoassays according to manufacturer's instructions (Osteomark® NTX serum, Wampole Laboratories). The intrinsic activity (lmaX) and potency (ICso) of the i-body on the serum NTx turnover can be described using an indirect response model. The indirect response model can be parameterized with the half-life of serum NTx (t112=0.693/kaut) and an i-body-mediated inhibition (Hill function) on the zero order production rate of NTx (K1n). The i-body can be assumed to inhibit K1n by means of a Hill equation parameterized with lmax, IC50 and a shape factor n. For each i-body a single set of PD parameters can be estimated.

### Example 11 Affinity Maturation of i-Bodies

In one example i-bodies can be modified only in the CDR1 and/or the CDR3 regions.

In a second example an error prone PCR strategy can be applied to the entire i-body polypeptide sequence with an aim of changing between 1-3 amino acids per mutant.

The i-body sequence can be mutagenized by error prone PCR using Taq DNA polymerase. (Leung et al (1989), Techniques 1; 11-15). Pools of mutated i-body cassettes will be isolated, cut with Sfil/Notl, cloned into the phagemid vector, and transformed into E. coli.

In both instances a mutant library is then screened using phage display. The selected mutants are then assessed for improvements in expression, binding affinity to RANK-L, in vitro and in vivo activity in various models as described in previous examples.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. (canceled)

2. A polypeptide which comprises a sequence derived from Domain 1 of NCAM comprising a scaffold region and CDR1 and CDR3 regions, wherein the CDR1 region of the sequence derived from Domain 1 of NCAM is replaced with a CDR 1 region comprising a sequence having at least 80% identity, or at least 90% identity to SEQ ID NO: 12; and wherein the CDR3 region derived from Domain 1 of NCAM is replaced with a CDR 3 region comprising a sequence having at least 80% identity, or at least 90% identity to SEQ ID NO: 13; and wherein the polypeptide binds to human RANK-L.

3. The polypeptide according to claim 2, wherein the CDR1 region derived from Domain 1 of NCAM is replaced with a CDR 1 region comprising a sequence having at least 95% identity, or at least 97% identity, or at least 98% identity, or at least 99% identity, or 100% identity to SEQ ID NO: 12.

4. The polypeptide according to claim 2, wherein the CDR3 region derived from Domain 1 of NCAM is replaced with a CDR 3 region comprising a sequence having at least 95% identity, or at least 97% identity, or at least 98% identity, or at least 99% identity, or 100% identity to SEQ ID NO: 13.

5. The polypeptide according to claim 2, wherein the scaffold region comprises a sequence at least 90% identical to a scaffold region defined by amino acids 1 to 26, 33 to 79 and 88 to 97 respectively of SEQ ID NO:1.

6. The polypeptide according to claim 2, wherein the positions of the CDR1 and CDR3 regions in the polypeptide respectively correspond to amino acids 27-32 and 80-87 of SEQ ID NO:1.

7. The polypeptide according to claim 2, wherein the scaffold region comprises a sequence which has at least 50%, or at least 60%, or at least 70%, or at least 80%, or at least 90%, or at least 95%, or at least 97%, or at least 98%, or at least 99% identity with SEQ ID NO:2.

8. The polypeptide according to claim 7, comprising a scaffold region comprising a sequence at least 80% identical to the sequence of SEQ ID NO: 11.

9. The polypeptide according to claim 2 which binds to human RANK-L with an affinity or avidity of less than or about 15 nM.

10. The polypeptide according to claim 2 wherein the polypeptide inhibits RANK-L induced osteoclastogenesis with an IC50 of less than about 5 nM.

11. The polypeptide according to claim 2 wherein the polypeptide comprises the sequence of SEQ ID NO: 11.

12. The polypeptide according to claim 2 which is PEGylated.

13. A nucleic acid molecule encoding the polypeptide according to claim 2.

14. A conjugate comprising the polypeptide according to claim 2 and an agent.

15. The conjugate according to claim 14, wherein the agent is a therapeutic agent, a toxin, a detectable label or an agent which extends the half-life of the polypeptide.

16. The conjugate according to claim 15 wherein the agent which extends the half-life of the polypeptide is a serum protein or an Fc portion of an immunoglobulin.

17. A multimer comprising two or more of the polypeptides according to claim 2 or a conjugate of the polypeptide and an agent.

18. A pharmaceutical composition comprising:
the polypeptide according to claim 2 or a conjugate of the polypeptide and an agent, or
a multimer comprising two or more of the polypeptides or the conjugate of the polypeptide and the agent; and
an acceptable carrier.

19. A method of treating a bone disorder comprising administrating to a subject in need thereof:
the polypeptide according to claim 2, or
a conjugate of the polypeptide and an agent, or
a multimer comprising two or more of the polypeptides or the conjugate of the polypeptide and the agent.

20. A method of manufacturing a medicament for treating a bone disorder, comprising providing:
a polypeptide according to claim 2, or
a conjugate of the polypeptide and an agent, or
a multimer comprising two or more of the polypeptides or the conjugate of the polypeptide and the agent.

21. A method of treating angiogenesis comprising administering to a subject in need thereof:
a polypeptide according to claim 2,
or a conjugate of the polypeptide and an agent, or
a multimer comprising two or more of the polypeptides or the conjugate of the polypeptide and the agent.
